(19) United States
(12) Patent Application Publication
(10) Pub. No.: US 2023/0225963 A1
(43) Pub. Date: Jul. 20, 2023

(54) THERMOGEL SUSTAINED-RELEASE MICROPARTICLE-BASED DELIVERY TO A PARANASAL AND/OR NASAL CAVITY

(71) Applicant: University of Pittsburgh- Of the Commonwealth System of Higher Education, Pittsburgh, PA (US)

(72) Inventors: Andrea L. Schilling, Pittsburgh, PA (US); Eric W. Wang, Pittsburgh, PA (US); Stella Lee, Pittsburgh, PA (US); Steven R. Little, Pittsburgh, PA (US)

(73) Assignee: University of Pittsburgh- Of the Commonwealth System of Higher Education, Pittsburgh, PA (US)

(21) Appl. No.: 18/008,799

(22) PCT Filed: Jun. 8, 2021
(86) PCT No.: PCT/US2021/036428

§ 371 Date: Dec. 7, 2022

Related U.S. Application Data
(60) Provisional application No. 63/036,739, filed on Jun. 9, 2020.

(51) Int. Cl. A61K 9/00; A61K 9/06; A61K 9/16; A61K 38/19; A61K 31/203; A61K 31/58

(52) U.S. Cl. CPC A61K 9/0043; A61K 9/06; A61K 9/1647; A61K 38/195; A61K 31/203; A61K 31/58

(57) ABSTRACT

A paranasal and/or nasal delivery system that includes a thermoresponsive gel; and a plurality of microparticles comprising a therapeutic amount of at least one paranasal and/or nasal condition-treating therapeutic agent, wherein the microparticles are included in the thermoresponsive gel.

This application claims the benefit of U.S. Provisional Application No. 63/036,739, filed Jun. 9, 2020, which is incorporated herein by reference in its entirety.

### BACKGROUND

Chronic rhinosinusitis (CRS) is an inflammatory disease of the sinonasal mucosa that affects nearly 12% of the US population (according to the CDC) and significantly reduces quality of life. CRS patients present with symptoms of increased nasal drainage, facial pain and pressure, congestion, and diminished olfaction persisting for over 12 weeks. Additionally, CRS patients report increased bodily pain and decreased social function, which contribute to a rating of general health status (health utility score) worse than that of patients with chronic obstructive pulmonary disorder or Parkinson's disease. In over 250,000 cases in the US annually, CRS patients with disease refractory to medical therapies elect to undergo functional endoscopic sinus surgery. In this procedure, inflamed tissue and bone are removed to reestablish sinus outflow tracts and improve access for topical maintenance therapy. However, there is need for improved methods of topical administration into the paranasal sinuses, even following surgery.

Currently prescribed intranasal corticosteroids, such as mometasone furoate, are beneficial for CRS symptom relief, however their local delivery to the sinuses is challenging. Nasal irrigation utilizing pots and squeeze bottles outperforms sprays in the ability to access to the sinus cavities, but requires high volumes, practiced technique, and daily dosing. The only local, controlled release devices for the paranasal sinuses are dissolvable stents (Propel® and Sinuva®), which are coated with mometasone furoate and inserted in one of the sinus cavities after surgery. Because the stents are designed to gradually degrade, local epithelial attenuation and cilia loss have been noted in the early response to stent placement. This inflammatory response can lead to clinical adverse events including crusting, granulation and scarring as well as nasal and ocular irritation. In one severe case, a stent was found to be extending through the skull base causing mental impairment. In this instance, the patient had undergone extensive surgery and eight stents were placed throughout the sinus cavities. The varying anatomy of the four sinuses (ethmoid, maxillary, frontal, and sphenoid) necessitates individual stents for each cavity and incorrect placement of the stents can lead to complications.

### SUMMARY

Disclosed herein in one embodiment is a paranasal and/or nasal delivery system comprising:

a thermoresponsive gel; and

a plurality of microparticles comprising a therapeutic amount of at least one paranasal and/or nasal condition-treating therapeutic agent,

wherein the microparticles are included in the thermoresponsive gel.

Disclosed herein in another embodiment is a method for treating a paranasal and/or nasal condition in a subject comprising administering to the subject in need thereof the paranasal and/or nasal delivery system, wherein the paranasal and/or nasal delivery system gels following administration.

The foregoing will become more apparent from the following detailed description, which proceeds with reference to the accompanying figures.

### BRIEF DESCRIPTION OF THE DRAWINGS

FIGS. 1A-1D. Bioactive mometasone furoate can be released from TEMPS for 4 weeks. (FIG. 1A) Size distribution and (inset) representative scanning electron micrograph, scale bar=10 μm, of drug-loaded MSs showing a smooth, spherical morphology with mean diameter of 7.8 μm. (FIG. 1B) Cumulative release of mometasone furoate from MSs (n=3) and (FIG. 1C) TEMPS, MSs embedded in hydrogel (n=5). Error bars represent mean±cumulative standard deviation, s. (FIG. 1D) Drug released from TEMPS after 28 days of aqueous incubation at 37° C. displays activity (EC50=5.13 pM) similar to that of drug prepared the day of the assay (inset, EC50=6.89 pM).

FIGS. 2A-2C. TEMPS is compatible for reversible apposition with the sinonasal epithelium. (FIG. 2A) Absorbance measurements at 415 nm as a function of increasing temperature for n=4 samples of thermogel and TEMPS showing that both undergo phase transition at 35° C. (FIG. 2B) The reversible temperature-responsive phase change of TEMPS demonstrated by repeated absorbance measurements at ambient temperature and 37° C. over 28 days. (FIG. 2C) Cytocompatibility of the PEG-pNIPAAm thermogel with RPMI 2650 sinonasal epithelial cell showing that cells remained viable after 24 hour incubation with the hydrogel. Cell viability was measured using PrestoBlue® reduction and % viability was calculated relative to positive and negative controls.

FIGS. 3A-3C. MicroCT imaging shows reduced opacification of right sinus cavity following application of m-TEMPS compared to v-TEMPS. (FIG. 3A) Disease was induced by creating a reversible obstruction of the left ostiomeatal complex for 2 weeks. Over the subsequent 11 weeks (disease induction) or 8 weeks (disease re-induction) chronic disease phenotype developed. Subjects were divided into 4 treatment groups, bilateral treatment was applied, and subjects were monitored for 4 weeks. (FIG. 3B) Representative coronal microCT section of the sinus cavities in soft tissue window and bone window (center). Opacification was measured using the bone window to define a region of interest and the median CT # of that region was recorded for 3 consecutive sections for each subject. (FIG. 3C) Change in CT # between disease and 2- or 4-weeks after treatment application on the right side with median CT # for each subject represented as individual symbols and error bars represent the mean±S.D. for treatment groups: no treatment (n=5), v-TEMPS (n=5), m-TEMPS (n=5), and daily nasal drops (n=3). Statistical significance was determined by one-way ANOVA with post hoc Wilcoxon Method testing, *** p<0.001, ** p<0.020

FIGS. 4A-4H. Evidence of iatrogenic trauma due to application of TEMPS was observed in right histopathology sections, but damage was reversed by local steroid delivery. Two sections from each subject were stained with H&E and scored by a blinded veterinary pathologist for (FIGS. 4A-4D) severity and involvement of (FIG. 4A) epithelial cell damage, (FIG. 4B) cilia damage, (FIG. 4C) inflammation of the submucosal glands, and (FIG. 4D) subepithelial edema, and (FIGS. 4E-4G) presence (1) or absence (0) of (FIG. 4E) granulocyte infiltrate, (FIG. 4F) granulocytes in the lumen, (FIG. 4G) involvement of the basement membrane, and (H) severity of epithelial hyperplasia. Individual symbols represent the score for each section and error bars represent the mean±S.D. for treatment groups: no treatment (n=4), v-TEMPS (n=3), m-TEMPS (n=4), and daily nasal drops (n=3). Statistical significance was determined by Chi-square test using Pearson's p-value, *p<0.050.

FIGS. 5A-5E. Intraocular pressure was not affected by 4 weeks of local steroid delivery from the retained system. (FIG. 5A) Mean bilateral IOP measurements for each subject are represented as individual symbols and error bars represent the mean±S.D. for the following timepoints and treatment groups: before treatment application (grey X, n=18), no treatment (n=5), v-TEMPS (n=5), m-TEMPS (n=5), and daily nasal drops (n=3). Solid and dotted lines denote the mean and range, respectively, of healthy New Zealand rabbit IOP values measured over a two-year period [16]. (FIG. 5B) A foreign material (yellow arrow) was recovered from the sinus cavity of a subject treated with TEMPS during post-mortem analysis. (FIG. 5C) The recovered material was visualized by SEM and spherical microspheres were observed, scale bar=10 μm. (FIG. 5D) Representative H&E section of a subject treated with TEMPS showed a foreign material apposed to the epithelium in the sinus cavity lumen that contains spherical holes, which are consistent with the expected appearance of TEMPS (40× magnification). (FIG. 5E) Percentage of subjects in each treatment group where TEMPS material was observed the H&E sections. Statistical significance was determined by repeated measures ANOVA with Tukey post-hoc testing, *p<0.050.

FIGS. 6A-6G. In vitro characterization of retinoic acid (RA) MS formulations. (FIGS. 6A-6D) MS morphology and release kinetics were affected by the weight of RA. (FIGS. 6A-6C) SEM images of MSs fabricated with RG 504H PLGA and loaded with (FIG. 6A) 5.0%, (FIG. 6B) 1.0%, and (FIG. 6C) 0.2% (w/w) RA (scale bar=10 μm) and (FIG. 6D) cumulative release profiles of these MS formulations. (FIG. 6E) Using PLGA with a higher lactic acid content (RG 755) or (FIG. 6F) larger molecular weight (RG 505) extended the release profile with kinetics influenced by RA weight or homogenization speed. (FIG. 6G) Bioactivity of RA released from MSs (RG 755, 1.0% RA, 6000 rpm) after 7 days incubation was comparable to RA reconstituted the day of the assay (“Drug Control”) (FIGS. 6D-6G) Error bars represent mean±S.D. (n=3).

FIGS. 7A-7B. Characterization of RA-TEMPS showing (FIG. 7A) cumulative drug release (n=3) and (FIG. 7B) gelation at 33° C.

FIG. 8. Release profile for rhCCL22 from Treg-TEMPS.

FIG. 9. Ciliated nasal epithelial samples incubated in thermogel for up to 24 h displayed a moderate to no effect on CBF. Error bars represent mean±S.D. for each group (18-25 min, n=4; 40-50 min, n=4; 2 h, n=3; 24 h, n=2). Statistical significance was determined by one-way ANOVA, *p<0.05. All other comparisons were not significant.

FIGS. 10A-10B. Images (top) and SEM (bottom) evaluating the texture and pore structure of (FIG. 10A) control and (FIG. 10B) 5% PEG2000 gels after freeze drying. Scale bar=100 μm.

FIGS. 11A-11B. Evaluation of the thermogel water content before freeze-drying and the residual moisture after freeze-drying. (FIG. 11A) The relative mass loss after freeze drying was used to determine the original water content (%). Error bars represent mean±S.D (n=11). Statistical significance was determined by ANOVA with Tukey post hoc testing, ***p<0.001, ns=not significant. (FIG. 11B) TGA curves showing the weight loss of freeze-dried thermogels as temperature is held at 120° C.

FIGS. 12A-12C. Swelling and gelation of freeze-dried formulations. (FIG. 12A) Photographs of freeze-dried gels and rehydration with simulated nasal fluid (SNF) over time. (FIG. 12B) Swelling ratio in water or SNF, (n=4). All differences were not significant. (FIG. 12C) Gelation kinetics of 5% PEG2000 gel rehydrated in SNF at indicated temperature measured by optical transmittance at 415 nm at 2- or 5-minute intervals.

FIGS. 13A-13H. Storage of FD-TEMPS and TEMPS for 3 weeks at ambient conditions altered the MS morphology, but not the release of bioactive mometasone furoate (MF). (FIGS. 13A-13D) SEM images of (FIG. 13A) MF-loaded MSs alone, (FIG. 13B) in FD-TEMPS, and following 3 weeks of storage in (FIG. 13C) FD-TEMPS or (FIG. 13D) TEMPS. Scale bar=5 μm. (FIGS. 13E-13G) Cumulative release profiles of MF from (FIG. 13E) FD-TEMPS immediately after preparation, (FIG. 13F) FD-TEMPS following storage, and (FIG. 13G) TEMPS following storage. (FIG. 13H) Bioactivity results of MF from prepared the day of the assay (Control) and MF released after 1 day of in vitro incubation of FD-TEMPS and TEMPS following storage.

FIG. 14. Transfer distance of thermogel and TEMPS in a hydrated or freeze-dried state. The lower, upper, and middle bars represent the minimum, maximum, and mean transfer distance (n=5). Statistical significance was determined by ANOVA, *** p<0.0001, * p<0.05.

### DETAILED DESCRIPTION

### Definitions and Abbreviations

The following explanations of terms and abbreviations are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements, unless the context clearly indicates otherwise.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. Other features of the disclosure are apparent from the following detailed description and the claims.

The disclosure of numerical ranges should be understood as referring to each discrete point within the range, inclusive of endpoints, unless otherwise noted. Unless otherwise indicated, all numbers expressing quantities of components, molecular weights, percentages, temperatures, times, and so forth, as used in the specification or claims are to be understood as being modified by the term “about.” Accordingly, unless otherwise implicitly or explicitly indicated, or unless the context is properly understood by a person of ordinary skill in the art to have a more definitive construction, the numerical parameters set forth are approximations that may depend on the desired properties sought and/or limits of detection under standard test conditions/methods as known to those of ordinary skill in the art. When directly and explicitly distinguishing embodiments from discussed prior art, the embodiment numbers are not approximates unless the word “about” is recited.

Although there are alternatives for various components, parameters, operating conditions, etc. set forth herein, that does not mean that those alternatives are necessarily equivalent and/or perform equally well. Nor does it mean that the alternatives are listed in a preferred order unless stated otherwise.

Definitions of common terms in chemistry may be found in Richard J. Lewis, Sr. (ed.), Hawley's Condensed Chemical Dictionary, published by John Wiley & Sons, Inc., 1997 (ISBN 0-471-29205-2).

In order to facilitate review of the various embodiments of the disclosure, the following explanations of specific terms are provided:

An “animal” refers to living multi-cellular vertebrate organisms, a category that includes, for example, mammals and birds. The term mammal includes both human and non-human mammals. Similarly, the term “subject” includes both human and non-human subjects, including birds and non-human mammals, such as non-human primates, companion animals (such as dogs and cats), livestock (such as pigs, sheep, cows), as well as non-domesticated animals, such as the big cats.

The term “biodegradable” means capable of being decomposed by a living organism, e.g., by a biological process.

The term “co-administration” or “co-administering” refers to administration of the delivery system disclosed herein with at least one other therapeutic agent or therapy within the same general time period, and does not require administration at the same exact moment in time (although co-administration is inclusive of administering at the same exact moment in time). Thus, co-administration may be on the same day or on different days, or in the same week or in different weeks. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent and/or lowers the frequency of administering the potentially harmful (e.g., toxic) agent. “Co-administration” or “co-administering” encompass administration of two or more active agents to a subject so that both the active agents and/or their metabolites are present in the subject at the same time. Co-administration includes simultaneous administration in separate compositions, administration at different times in separate compositions, or administration in a composition in which two or more active agents are present.

A “gel” is a colloidal system comprising a solid three-dimensional network within a liquid. By weight, a gel may be primarily liquid, but behaves like a solid due to a three-dimensional network of entangled and/or crosslinked molecules of a solid within the liquid. From a rheological perspective, a gel has a storage modulus G′ value which exceeds that of the loss modulus G″. The storage modulus and loss modulus can be determined with a rheometer.

A “thermoresponsive gel” is a three-dimensional network of polymeric chains that are capable of absorbing and retaining molecules (e.g., water, polar solvents, non-polar solvents, drugs in liquid form, or the like) in their three-dimensional networks, wherein the gel undergoes a change from a hydrophilic state to a hydrophobic state as temperature changes. Thermoresponsive gel-forming polymeric chains may comprise one or more hydrophilic functional groups in their polymeric structures, such as amino (—NH₂), hydroxyl (—OH), amide (—CONH—, —CONH₂), sulfate (—SO₃H), or any combination thereof, and can be natural-, or synthetic-polymeric-based networks. In some embodiments, the polymeric chains can comprise a plurality of the same monomeric units. In other embodiments, the polymeric chains can comprise a plurality of different monomeric units. “Inhibiting” refers to inhibiting the full development of a disease or condition. “Inhibiting” also refers to any quantitative or qualitative reduction in biological activity or binding, relative to a control. As used herein with respect to chronic rhinosinusitis, inhibiting refers to reducing or eliminating signs of inflammation, such as purulence, edema, nasal polyps, radiographic mucosal changes, and/or symptoms of inflammation such as facial pain/pressure/fullness, nasal drainage/blockage/obstruction/congestion, and/or diminished sense of smell.

“Lower Critical Solution Temperature” (LCST) refers to a critical temperature at or above which a thermoresponsive gel can undergo a change from its hydrophilic state to its hydrophobic state, or vice versa. In some embodiments, a thermoresponsive gel is hydrated below its LCST, and therefore is hydrophilic. In some embodiments, a thermoresponsive gel is at least partially dehydrated above its LCST, and therefore is insoluble and hydrophobic. In some embodiments, LCST of linear thermo-responsive polymers is determined using cloud point (CP), and is generally used for physically crosslinked polymers. Cloud point refers to the temperature at the outset of cloudiness, the temperature at inflection point of a transmittance curve, or the temperature at a defined transmittance. The cloud point can be affected by many structural parameters of the thermoresponsive gel like the hydrophobic content, architecture of the thermoresponsive gel, molar mass of the thermoresponsive gel, or any combinations thereof.

“Microparticle,” as used herein, unless otherwise specified, generally refers to a particle of a relatively small size, but not necessarily in the micron size range; the term is used in reference to particles of sizes that can be, for example, administered to the nasal cavity and/or to the sinus(es) in the form of biodegradable polymers that encapsulate a therapeutic agent for controlling the release profile of the encapsulated therapeutic agent. In certain embodiments, microparticles specifically refers to particles having a diameter from 0.2 to 50 microns, such as from 0.2 to 25 microns. In one embodiment, the particles have a diameter from 0.2 to 10 microns, 0.2 to 5 microns, or 0.2 to 3 microns. As used herein, the microparticle encompasses microspheres, microcapsules, microparticles, microrods, nanorods, nanoparticles, or nanospheres unless specified otherwise. A microparticle may be of composite construction and is not necessarily a pure substance or a pure polymer; it may be spherical or any other shape.

A “polymer” is a molecule of repeating structural units (e.g., monomers) formed via a chemical reaction, i.e., polymerization. A “copolymer” is a polymer formed from polymerization of two or more different monomers. Simultaneous polymerization of two or more different monomers generally produces a “random copolymer.” Unless otherwise specified, polymer molecular weights provided herein are weight average molecular weight, M_{w}.

A “therapeutically effective amount” refers to a quantity of a specified agent sufficient to achieve a desired effect in a subject being treated with that agent. Ideally, a therapeutically effective amount of an agent is an amount sufficient to inhibit or treat the disease or condition without causing a substantial cytotoxic effect in the subject. The therapeutically effective amount of an agent will be dependent on the subject being treated, the severity of the affliction, and the manner of administration of the therapeutic composition. For example, a “therapeutically effective amount” may be a level or amount of agent needed to treat a nasal or sinus condition, or reduce or prevent nasal or sinus injury or damage without causing significant negative or adverse side effects to the nasal or sinus cavity.

“Treatment” refers to a therapeutic intervention that ameliorates a sign or symptom of a condition (e.g., a disease or pathological condition) after it has begun to develop. As used herein, the term “ameliorating,” with reference to a condition, refers to any observable beneficial effect of the treatment. The beneficial effect can be evidenced, for example, by a delayed onset of clinical symptoms of the condition in a susceptible subject, a reduction in severity of some or all clinical symptoms of the condition, a slower progression of the condition, an improvement in the overall health or well-being of the subject, or by other parameters well known in the art that are specific to the particular condition. The phrase “treating a condition” refers to inhibiting the full development of a condition, for example, in a subject who is at risk for a condition. “Preventing” a condition refers to prophylactic administering a composition to a subject who does not exhibit signs of a condition or exhibits only early signs for the purpose of decreasing the risk of developing a pathology or condition, or diminishing the severity of a pathology or condition.

“Pharmaceutical compositions” are compositions that include an amount (for example, a unit dosage) of one or more of the disclosed compounds together with one or more non-toxic pharmaceutically acceptable additives, including carriers, diluents, and/or adjuvants, and optionally other biologically active ingredients. Such pharmaceutical compositions can be prepared by standard pharmaceutical formulation techniques such as those disclosed in Remington's Pharmaceutical Sciences, Mack Publishing Co., Easton, Pa. (19th Edition).

The terms “pharmaceutically acceptable salt” refers to salts prepared by conventional means that include salts, e.g., of inorganic and organic acids, including but not limited to hydrochloric acid, hydrobromic acid, sulfuric acid, phosphoric acid, methanesulfonic acid, ethanesulfonic acid, malic acid, acetic acid, oxalic acid, tartaric acid, citric acid, lactic acid, fumaric acid, succinic acid, maleic acid, salicylic acid, benzoic acid, phenylacetic acid, mandelic acid and the like. These salts may be prepared by standard procedures, for example by reacting the free acid with a suitable organic or inorganic base. Any chemical compound recited in this specification may alternatively be administered as a pharmaceutically acceptable salt thereof. “Pharmaceutically acceptable salts” are also inclusive of the free acid, base, and zwitterionic forms. Descriptions of suitable pharmaceutically acceptable salts can be found in Handbook of Pharmaceutical Salts, Properties, Selection and Use, Wiley VCH (2002). Such salts are known to those of skill in the art. For additional examples of “pharmacologically acceptable salts,” see Berge et al., J. Pharm. Sci. 66:1 (1977). The pharmaceutically acceptable acid addition salts can conveniently be obtained by treating the base form with such appropriate acid. Appropriate acids comprise, for example, inorganic acids such as hydrohalic acids, e.g. hydrochloric or hydrobromic acid, sulfuric, nitric, phosphoric and the like acids; or organic acids such as, for example, acetic, propanoic, hydroxyacetic, lactic, pyruvic, oxalic (i.e. ethanedioic), malonic, succinic (i.e. butanedioic acid), maleic, fumaric, malic (i.e. hydroxybutanedioic acid), tartaric, citric, methanesulfonic, ethanesulfonic, benzenesulfonic, p-toluenesulfonic, cyclamic, salicylic, p-aminosalicylic, pamoic and the like acids. Conversely said salt forms can be converted by treatment with an appropriate base into the free base form.

### Paranasal or Sinus Delivery System

The delivery system includes microparticles dispersed in a thermoresponsive gel, the microparticles comprising at least one therapeutic agent. In certain embodiments, the microparticles also include a polymer that provides a sustained drug release profile. In certain embodiments, the microparticles also include a ceramic that provides a sustained drug release profile. In certain embodiments, the microparticles are in the form of a micronized therapeutic agent that has low solubility and dissolves slowly. The system provides local and sustained release of a therapy to the sinus and/or nasal cavities. In certain embodiments, the system provides local and sustained release of a therapy to the sinus cavities to improve patient outcomes following functional endoscopic sinus surgery for the treatment of chronic rhinosinusitis.

In certain embodiments, the delivery system is referred to herein as “TEMPS” (Thermogel, Extended Release, Microparticle-based-delivery to the Paranasal Sinuses).

In certain embodiments, the delivery system includes a thermo-responsive acrylamide-based hydrogel (thermogel) and extended-release polylactic-co-glycolic acid (PLGA) microparticles (particularly microspheres) encapsulating a therapeutic agent. In certain embodiments, the therapeutic agent is corticosteroid mometasone furoate.

The degradable microparticles can be tuned to provide sustained drug release, thus avoiding daily dosing. In certain embodiments, the delivery system releases a therapeutic for up to four weeks. The microparticles are embedded in the thermogel, which localizes them to the paranasal sinuses for long-acting treatment. The thermogel is tuned to undergo a phase change from a flowable liquid at room temperature to a gel at body temperature, so that it can be applied by a physician after surgery using a transnasal catheter and upon contact with the sinus and/or nasal tissue, the material will gel and conform to the unique anatomy of the sinuses. For removal, the phase change can be reversed by room temperature saline irrigation. The system can be reapplied as needed to continue treatment.

A study testing the performance of TEMPS in rabbits showed that the system is well-tolerated, retained for four weeks, does not cause an increase in ocular pressure, and reduces sinonasal inflammation. The compositions disclosed herein offer the flexibility of encapsulating different drugs in the polymeric microspheres and broader implications for treatments of various inflammatory or allergic sinonasal conditions. The system disclosed herein conforms to the sinonasal mucosa, inspired by the native mucus layer, addressing the issues with existing devices (e.g., stents) and is much better suited for long-term application.

The polymers for the microparticles may be biodegradable polymers so long as they are biocompatible (i.e., do not produce adverse effects (toxicity, irritation, etc.) when administered to a subject). Preferred biodegradable polymers are polyhydroxyacids such as polylactic acid and copolymers thereof. Illustrative polymers include polyglycolide (PGA), polylactic acid) (PLA), and poly(lactic-co-glycolic acid) (PLGA). Another class of approved biodegradable polymers is the polyhydroxyalkanoates. The percent loading of a therapeutic agent may be increased by “matching” the hydrophilicity or hydrophobicity of the polymer to the agent to be encapsulated. In some cases, such as PLGA, this can be achieved by selecting the monomer ratios so that the copolymer is more hydrophilic for hydrophilic drugs or less hydrophilic for hydrophobic drugs.

In some embodiments, the polymer is a PLGA copolymer. The weight average molecular weight of PLGA is from 4 kDa to 80 kDa, such as from 4 kDa to 50 kDa, or from 4 kDa to 15 kDa. The ratio of lactide to glycolide is from about 75:25 to about 50:50. In one embodiment, the ratio is 75:25. The PLGA may be ester-terminated or acid-terminated. The molecular weight of the PLGA may control the degradation rate of the microparticles and subsequent drug release kinetics. Illustrative polymers include, but are not limited to, poly(D,L-lactic-co-glycolic acid) (PLGA, 75:25 lactic acid to glycolic acid ratio, MW_{w}=4-15 kDa, referred to as 752H); poly(D,L-lactic-co-glycolic acid) (PLGA, 50:50 lactic acid to glycolic acid ratio, MW_{w}=24-38 kDa, referred to as 503H); poly(D,L-lactic-co-glycolic acid) (PLGA, 50:50 lactic acid to glycolic acid ratio, MW_{w}=38-54 kDa, referred to as 504H); poly(D,L-lactic-co-glycolic acid) (PLGA, 50:50 lactic acid to glycolic acid ratio, MW_{w}=38-54 kDa, referred to as 504); poly(D,L-lactic-co-glycolic acid) (PLGA, 75:25 lactic acid to glycolic acid ratio, MW_{w}=25 kDa, referred to as RG755); and poly(D,L-lactic-co-glycolic acid) (PLGA, 50:50 lactic acid to glycolic acid ratio, MW_{w}=7-17 kDa, referred to as RG502H). In certain examples, the polymer is PLGA 752H. In certain examples, the polymer is PLGA 752. In certain examples, the polymer is PLGA 503H. In certain examples, the polymer is PLGA 503. In certain examples, the polymer is PLGA 504H. In certain examples, the polymer is PLGA 504.

In some embodiments, the polymer is PLA. The weight average molecular weight may be from 20-80 kDa, such as from 20-50 kDa or 20-30 kDa.

In some embodiments, polymer chains in the thermoresponsive gel are not crosslinked Absence of crosslinking removes barriers to diffusion. In certain embodiments, a wide range of molecular weights as described above and/or polymer concentrations are effective so long as the concentration provides a thermoresponsive gel that forms a gel below the lower critical solution temperature.

In some embodiments, the amount of therapeutic agent loaded into the microparticles may be from 5 ng to 100 μg, such as 5 to 1000 ng or 1 to 100 ng per milligram of microparticles. In certain embodiments, the amount of therapeutic agent loaded into the microparticles is 25 to 100 μg therapeutic agent per mg of microparticles.

The therapeutic agent-loaded microparticles may have a volume average diameter of 200 nm to 50 μm, such as 1 μm to 25 μm. In certain embodiments, the microparticles do not have a volume average diameter of 50 μm or greater since such larger particles are difficult to eject from a catheter.

The microparticles are dispersed in a thermoresponsive gel. Advantageously, the selected thermoresponsive gel has a lower critical solution temperature (LCST) below body temperature. The thermoresponsive gel remains fluid below physiological temperature (e.g., 37° C. for humans) or at or below room temperature (e.g., 25° C.), solidifies (into a hydrogel) at physiological temperature, and is biocompatible. For example, the thermoresponsive gel may be a clear liquid at a temperature below 34° C. which reversibly solidifies into a gelled composition at a temperature above 34° C. Generally, the LCST-based phase transition occurs upon warming in situ as a result of entropically-driven dehydration of polymer components, leading to polymer collapse. Various naturally derived and synthetic polymers exhibiting this behavior may be utilized. Natural polymers include elastin-like peptides and polysaccharides derivatives, while notable synthetic polymers include those based on poly(N-isopropyl acrylamide) (PNIPAAm), poly(N,N′-dimethylacrylamide-co-N-phenylacrylamide), poly(glycidyl methacrylate-co-N-isopropylacrylamide), poly(ethylene oxide)-b-poly(propylene oxide)-b-poly(ethylene oxide), poly(ethylene glycol)-polyester copolymer, and amphiphilic block copolymers. In some embodiments, the thermoresponsive gel is PNIPAAm. The structure of PNIPAAm, containing both hydrophilic amide bonds and hydrophobic isopropyl groups, leads to a sharp phase transition at the LCST. Studies suggest that the average number of hydrating water molecules per NIPAAm group falls from 11 to about 2 upon the hydrophobic collapse above the LCST (32-34° C.).

In some embodiments, the thermoresponsive gel is non-biodegradable, e.g., PNIPAAm or a copolymer of N-isopropylacrylamide and at least one acrylic and/or methacrylic monomer. In some embodiments, the thermoresponsive gel is a mixture of N-isopropylacrylamide and a hydrophilic polymer (e.g., polyethylene glycol (PEG)). In certain embodiments, the MW_{w} of the polymer or copolymer may be 5-20,000 kDa. In certain embodiments, the mol % for the N-isopropylacrylamide monomer in the copolymerization reaction may be 50-99 mol %. Illustrative acrylic monomers include an acrylate such as an alkyl acrylate (e.g., methyl acrylate, ethyl acrylate, butyl acrylate or 2-ethylhexyl acrylate), an acrylamide; or an acrylic acid or salt (e.g., 2-ethylacrylic acid, 2-propylacrylic acid, N-acryloxysuccinimide). Illustrative methacrylic monomers include a methacrylate (e.g., 2-hydroxymethacrylate, hydroxyethyl methacrylate, butyl methacrylate, methyl ether methacrylate or methyl methacrylate); a methacrylamide; or a methacrylic acid or salt. In certain embodiments, the acrylate monomer or methacrylate monomer may be modified with poly(ethylene glycol) to provide a co-poly(ethylene glycol) acrylate or co-poly(ethylene glycol) methacrylate prior to reaction with the N-isopropylacrylamide monomer. Acrylated PEG monomer(s) can be added in an amount of 1-15 mol %. In certain embodiments, PEG is an additive for mixing with N-isopropylacrylamide. The PEG may have a molecular weight of 200 Da to 100,000 Da.

In other embodiments, the thermoresponsive gel is biodegradable. For example, biodegradable NIPAAm-based polymers can be made by conjugating the PNIPAAm with natural biodegradable segments such as MMP-susceptible peptide, gelatin, collagen, hyaluronic acid and dextran. Copolymers formed from NIPAAm and monomers with degradable side chains comprise another category of NIPAAm-based bioabsorbable, thermoresponsive gels.

The delivery system disclosed herein may provide for sustained release of a therapeutic agent. For example, the sustained release may be over a period of at least 365 days, such as at least 60 days, at least 30 days, or at least 1 day. In certain embodiments, the system disclosed herein can effectively provide up to at least four weeks, more particularly up to three weeks, and most particularly up to one weeks of sustained release of a bioactive steroid or other therapeutic agent. The therapeutic agent release can be linear or non-linear (single or multiple burst release). In certain embodiments, the therapeutic agent may be released without a burst effect. For example, the sustained release may exhibit a substantially linear rate of release of the therapeutic agent in vivo over a period of at least 365 days, such as at least 60 days, at least 30 days, or at least 1 day. By substantially linear rate of release it is meant that the therapeutic agent is released at a rate that does not vary by more than about 20% over the desired period of time, more usually by not more than about 10%. It may be desirable to provide a relatively constant rate of release of the therapeutic agent from the delivery system over the life of the system. For example, it may be desirable for the therapeutic agent to be released in amounts from 20 to 250 μg per day, more particularly 20 to 200 μg per day, for the life of the system. However, the release rate may be either increased or decreased depending on the formulation of the polymer microparticle and/or thermoresponsive gel.

In certain embodiments, the therapeutic agent release is dependent on degradation of the polymer microparticles. As the polymer (e.g., PLGA) chains break up, the therapeutic agent can diffuse out of the initial polymer microparticle matrix where it will eventually reach the thermoresponsive gel matrix. Diffusion through the thermoresponsive gel is significantly faster than degradation of the polymer. Thus the limiting factor in therapeutic agent release is degradation of the polymer.

A therapeutically effective dose of therapeutic agent may be within a range of 1 pg to 400 μg per day. In the case of mometasone, the dose may be 50 μg to 400 μg per day. The delivery system is formulated such that each dose will release a therapeutically effective amount of therapeutic agent each day.

The delivery system may be administered to any paranasal and/or nasal mucosal surface. At the end of the desired administration period, the gelled system can be removed from the cavity (for example, via flushing out). In certain embodiments, the thermoresponsive gel may be biodegradable so that there is no need to remove the gelled system (this embodiment may be most useful for treating an acute condition).

The therapeutic agent may be an agent that inhibits, ameliorates and/or inhibits a paranasal and/or nasal condition. Illustrative therapeutic agents include an intranasal corticosteroid (e.g., mometasone furoate, fluticasone propionate, ciclesonide, fluticasone furoate, dexamethasone, flunisolide, triamcinolone acetonide, beclomethasone dipropionate, budesonide), an antibiotic, a biologic (e.g., CCL22, IL-2, TGF-β), rapamycin, a retinoid (e.g., retinoic acid), or a combination thereof.

Illustrative conditions that may be treated by the system disclosed herein include chronic rhinosinusitis, with and without nasal polyps, acute rhinosinusitis, recurrent acute rhinosinusitis, allergic rhinitis and olfactory disorders.

In certain embodiments, administration of the delivery system promotes regeneration of ciliated nasal epithelium.

In certain embodiments, the delivery system disclosed herein is administered topically to at least one paranasal and/or nasal cavity. For example, the delivery system may be administered transnasally via a catheter for delivery to the desired tissue. The system may be applied by entering through the naris (nostril) using a catheter and endoscope for visualization. The system can be applied by actuating a syringe or other vessel containing the system for its application or dispersion in the sinus or nasal cavities. The system can be applied as a coating along the sinonasal epithelium, either targeted to specific areas of inflammation or distributed throughout the sinus cavities for widespread treatment.

In certain embodiments, the microparticles are mixed with the thermoresponsive gel immediately prior to administering to the subject.

In certain embodiments, the delivery system may also include a physiologically acceptable vehicle, carrier, excipient, binder, preservative, stabilizer, flavor, or the like. The delivery system may be formulation into a unit dosage form. The term “unit dosage form” refers to physically discrete units suitable as unitary dosages for human subjects and other mammals, each unit containing a predetermined quantity of active material calculated to produce the desired therapeutic effect, in association with a suitable pharmaceutical excipient.

In certain embodiments, the delivery system may be in a freeze-dried form. In certain embodiments, the delivery system is in a lyophilized powder form.

### EXAMPLES

### Microsphere Fabrication and Characterization

Drug-loaded polymer microspheres were fabricated using a standard single emulsion procedure. 200 mg PLGA (0.32-0.44 dl/g, Sigma, St. Louis, Mo.) and 10 mg mometasone furoate (AcrosOrganics, Thermo Fisher Scientific, Waltham, Mass.) were mixed with 4 mL dichloromethane. The drug/polymer solution was homogenized (Silverson L4RT-A, East Longmeadow, Mass.) in 60 mL 2% poly(vinyl alcohol) (PVA, MW ˜25 kDa, 98% hydrolyzed, PolySciences, Warrington, Pa.) at 5000 rpm for 1 min. The emulsion was then mixed with 80 mL 1% PVA and stirred at 600 rpm for 3 h for solvent evaporation. Microspheres were collected, washed 4× with MilliQ water, flash-frozen with liquid nitrogen, and lyophilized (VirTis Benchtop K freeze dryer, Gardiner, N.Y. operated at 100 mTorr) for 48 h before storage at −20° C. Samples of MSs were visualized by scanning electron microscopy (SEM) (JEOL JSM-6510LV/LGS SEM, Japan) following gold-palladium sputter coating (Denton Sputter Coater, Moorestown, N.J.). Size distribution of MSs was determined by volume impedance measurements (Multisizer 3 Coulter Counter, Beckman Coulter, Indianapolis, Ind.).

Drug encapsulation was evaluated by dissolving 5 mg mometasone-loaded or blank MSs in 2 mL dimethyl sulfoxide (DMSO). The drug concentration was quantified by absorbance measurements at 262 nm using a UV-Vis spectrophotometer (Molecular Devices, Sunnyvale, Calif.). Encapsulation efficiency was calculated as the ratio of mometasone measured in the dissolved MSs relative to the amount that was initially added during fabrication and normalized based on MS yield.

### Hydrogel Fabrication and Characterization

Thermoresponsive hydrogel was prepared using reagents from Sigma-Aldrich (St. Louis, Mo.) and methods described by Bellotti et al. J. Mater. Chem. B, vol. 7, no. 8, pp. 1276-1283, 2019. Briefly, 1004 of 200 Da poly(ethylene glycol) (PEG) was added to 100 mg of NIPAAm. The aqueous free radical polymerization reaction was initiated by the addition of ammonium persulfate and tetramethylethylenediamine. Following overnight polymerization at 4° C., the hydrogel was washed 10× with MilliQ water warmed to 40-50° C. to remove unreacted monomer. The resulting hydrogel was stored at room temperature.

The thermo-reversible nature of TEMPS was evaluated, as well as the system's lower critical solution temperature (LCST) and cytocompatibility of the pNIPAAm gel as previously described in Bellotti et al. Reversibility of the sol-gel transition was demonstrated in vitro, whereby TEMPS and its individual gel and MS components were incubated in a clear bottom, 96-well plate in excess an aqueous salt solution of simulated nasal fluid. At weekly intervals, absorbance was measured at 37° C. and 415 nm. Then the plate was cooled to room temperature and absorbance was measured again. The aqueous solution was replenished as needed and the plate was sealed during incubation to prevent evaporation.

For cytocompatibility measurements, a sinonasal epithelial cell line, RPMI 2650 (ATCC® CCL30™) was cultured in Eagle's Minimum Essential Medium (American Type Culture Collection, Manassas, Va.) supplemented with 10% FBS. Cells were seeded at 20,000 cells per well in a 96-well plate. Following incubation for 24 hours with the hydrogel, 10% PrestoBlue® viability reagent (ThermoFisher Scientific, Waltham, Mass.) was added. After 3 hours incubation, reduction of PrestoBlue reagent was measured by fluorescence readings using an excitation filter of 540 nm and an emission filter of 580 nm. The mean and standard deviation of fluorescence values (F) were determined (n=6) and viability was calculated according to the following equation:

$$\% Viability = 100 \times [ 1 - \frac{( {F}_{positive control} - {F}_{gel sample} )}{( {F}_{positive control} - {F}_{negative control} )} ]$$

### In Vitro Drug Release and Bioactivity

In vitro release kinetics of mometasone furoate were evaluated using methods adapted from Ammar et al “Local delivery of mometasone furoate from an eluting endotracheal tube,” J. Control. Release, vol. 272, no. 272, pp. 54-61, 2018. To quantify release from MSs only, 10 mg MSs were incubated in 1 mL 2% sodium deoxycholate in water on a roto-shaker at 37° C. (n=3). At regular time intervals, MS suspensions were centrifuged, the supernatant was collected and diluted 1:3 in methanol, and the absorbance was measured at 248 nm. Absorbance of releasate from blank MSs was measured and subtracted at each timepoint. Drug release from TEMPS was determined using a similar procedure, with the exception that 10 mg MSs were suspended in 1004 pNIPAAm hydrogel (n=5).

Throughout the in vitro release assay, releasate samples were diluted with DMSO and stored at −20° C. for bioactivity measurements. Following the vendor protocol for the Human Glucocorticoid Receptor (NR3C1, GR) Reporter Assay (Indigo Biosciences, State College, Pa.), release samples from TEMPS between day 25 and 28 of incubation were tested for their ability to bind to the engineered cells and induce luciferase expression, indicative of the presence of bioactive drug. As a control, drug was solvated in DMSO the day of the assay to compare the bioactivity of fresh (n=1-2 per dilution) and released drug (n=3 per dilution).

### Animal Model

This study evaluating TEMPS in male and female New Zealand rabbits (2-4 kg) was approved by the Institutional Animal Care and Use Committee (IACUC) at the University of Pittsburgh. When necessary, animals were anesthetized with ketamine (10-50 mg/kg IM) and xylazine (2-5 mg/kg SQ). Lidocaine hydrochloride was applied to the nares to reduce sensation for endoscopy. Following all procedures, antisedan (5 mg/kg IV) was dosed for xylazine reversal and rabbits were given SQ saline (10 mL/kg) for higher anesthesia doses. If pain management following procedures was necessary, meloxicam was dosed for 1-3 days (0.2 mg/kg PO or SQ).

### Disease Induction and MicroCT Imaging

A reversible obstruction of the left ostiomeatal complex was created to induce disease. Animals were anesthetized (ketamine, 35 mg/kg IM and xylazine, 5 mg/kg SQ) and the nasal cavity was visualized with a 1.9 mm 0° endoscope (Karl Storz, Tuttlinggen, Germany). A sterile polyvinyl alcohol sponge (aseptically trimmed to approximately 12×3×1 mm) was inserted through the left naris and packed in the opening of the sinus ostia, lateral to the middle meatus, as previously described. Two weeks later, the sponge was removed. Over the subsequent 11 weeks, disease phenotype progressed towards a chronic pathology.

Opacification of the sinus cavities was monitored by skull micro computed tomography (CT) imaging using a Fidex system (Animage, Pleasanton, Calif.). For subjects that did not show signs of opacification after the initial chronic disease induction, disease was re-induced as follows. Three sponges were inserted to ensure full blockage of the left sinus ostia for 2 weeks. Eight weeks after the sponge removal, microCT imaging was performed to assess disease re-induction.

### Treatment Application

Following chronic disease induction, animals were assigned to groups for bilateral treatment. A one-time application of TEMPS was performed by percutaneously injecting the material into the sinus cavities using an 18 G needle. Approximately 20 mg of blank MSs or mometasone-loaded MSs were suspended in 0.4 mL of hydrogel and injected into the left and right sinus cavities for the vehicle-only TEMPS (v-TEMPS) or mometasone-loaded TEMPS (m-TEMPS) groups, respectively. A no treatment group served as a negative control that also received the one-time percutaneous punctures without material injection. Similarly, a positive control treatment group received the one-time bilateral punctures without injection and then daily nasal drops for 4 weeks of 31.25 μg of steroid in 1004 (Mometasone Furoate Nasal Spray, Apotex Inc., Toronto, Ontario, diluted with PBS) to the left and right nares via a 0.5-inch 22 G flexible PTFE dispensing needle.

### In Vivo Measurements

Bilateral intraocular pressure (IOP) was measured at 4 timepoints: baseline, disease, 2-weeks and 4-weeks after treatment application. To control for diurnal variations, IOP measurements were performed in the morning by the same technician on unrestrained, alert animals using a TonoVet rebound tonometer (Icare, Finland).

Additionally, at these 4 timespoints, bilateral sinus opacification was evaluated using microCT imaging. Using OsiriX Lite (Pixmeo SARL, Switzerland), the left and right sinus cavities were identified in a coronal view on three consecutive 155.79 μm thickness scans near the mid-point of the middle turbinate. The image contrast and brightness was adjusted so that only calcified tissue was visible (window level ˜800, window width ˜1400) and a region of interest was defined by tracing a polygon along the inner edge of the cavity wall, excluding calcified bone. The median CT # of this defined region, as calculated by the software, was recorded for analysis by an investigator blinded to the subject ID and group for measurements at 2- and 4-weeks after treatment application.

### Histologic Preparation and Assessment

At the conclusion of the study, anesthetized animals were euthanized with pentobarbitol (100 mg/kg IV) and tissue was prepared for histological evaluation. Maxilla bones were harvested and fixed by immersion in 10% neutral buffered formalin for 1 week. Following fixation, maxillary bones were transferred to 10% formic acid for decalcification. After 1 week, the decalcification solution was tested every other day for end-point determination using 5 mL of the used decalcification solution combined with 5 mL ammonium hydroxide and 5 mL ammonium oxalate. If the test solution was cloudy, the tissue was placed in new formic acid until the next test day. Once the test solution yielded a clear solution, sections were cut, placed in cassettes, dehydrated in a graded series of alcohol, and embedded in paraffin blocks. Paraffin-embedded section were cut to 4 μm using a microtome, mounted on glass slides, and stained with routine hematoxylin and eosin (H&E). Inflammatory scoring criteria were developed for analysis of maxillary sinus sections. Scoring analysis was performed in a blinded manner by a board-certified veterinary pathologist.

Four histopathology items were scored for their relative involvement in the histology sections: 0=none, 1=up to ⅓, 2=⅓ to ⅔ and 3=⅔ to diffuse. Involvement scores were multiplied by severity scores from 0-3 (none, mild, moderate, severe) for epithelial cell damage, subepithelial edema and inflammation of the submucosal glands and 0-2 (normal, disrupted, loss) for cilia damage. Epithelial hyperplasia severity only was scored from 0-3 (none, mild, moderate, severe). The remaining 3 items, basement membrane, granulocyte tissue infiltrate and granulocytes in the lumen, were scored for 0 or 1 for absence or presence, respectively.

### Statistical Analyses

Statistical analyses were performed with SAS JMP® Pro 14 (Cary, N.C.) and GraphPad Prism v7 (San Diego, Calif.). Descriptive statistics were used to describe the mean and standard deviations (S.D.) and values are reported as the mean±S.D. For the in vitro bioactivity assay, ED50 values and 95% confidence intervals (CI) were determined by nonlinear 3- or 4-parameter logistic regression using GraphPad. Using JMP, comparisons among groups with continuous data were performed by one-way ANOVA. After assessing the variances by Levene's test, Wilcoxon Method (unequal variance) or Tukey (equal variance) post-hoc testing was performed. For nominal data, comparisons among groups were performed by Chi-square testing using Pearson's p-values. For all analyses, p<0.05 was considered as significant.

### Results

### TEMPS Provides Extended Release of Bioactive Steroid in a Thermoresponsive and Cytocompatible System

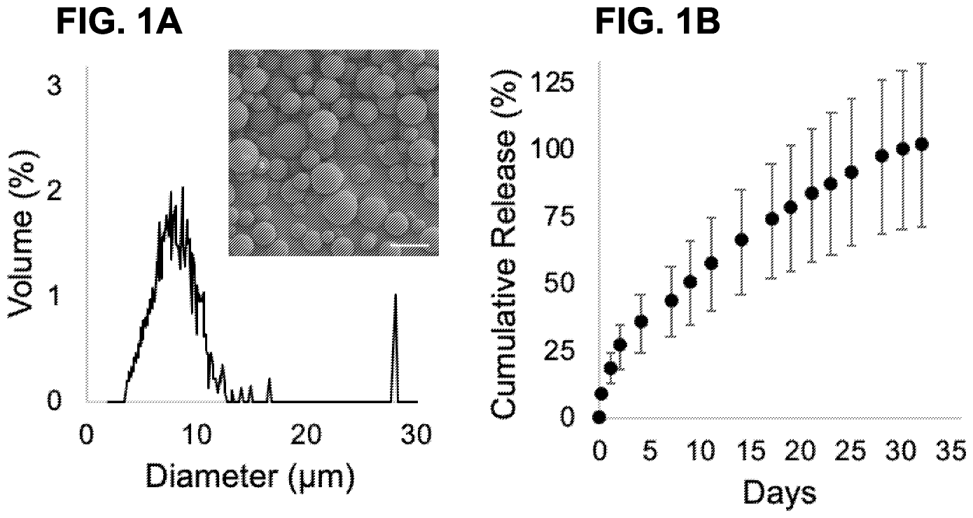

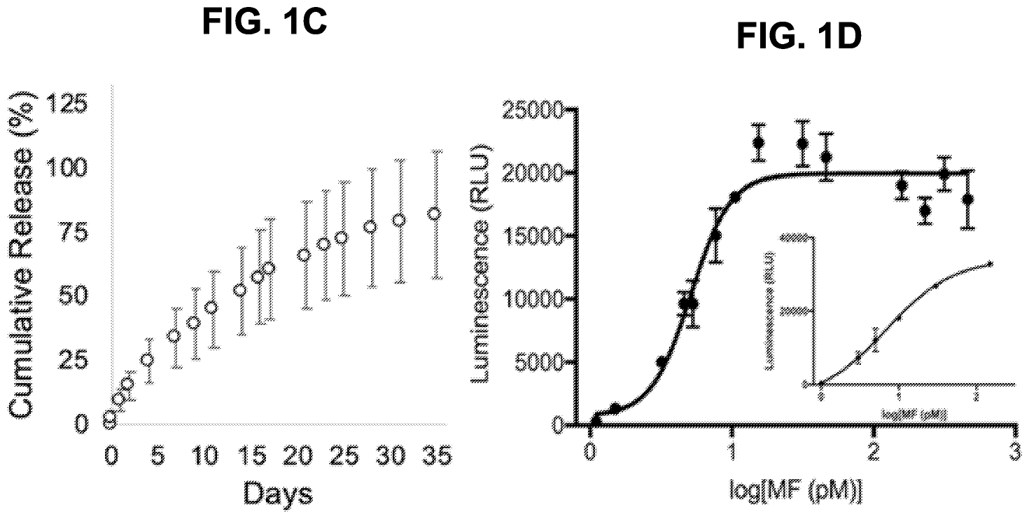

The combined thermogel and polymer microparticle system, or TEMPS, was engineered to provide extended release of bioactive steroid for multiple weeks. Steroid, mometasone furoate, was encapsulated in PLGA MSs that had a spherical morphology with mean diameter of 7.8±2.9 μm, which was measured by volume impedance (FIG. 1A). Approximately 44 μg of mometasone per mg MS was encapsulated, with a loading efficiency of 88%. The sustained release of mometasone for 4 weeks was demonstrated in vitro from both MSs (FIG. 1B) and TEMPS (FIG. 1C). Additionally, the bioactivity of released mometasone from TEMPS was similar to fresh drug. TEMPS releasate that was collected between day 25 and 28 had an EC50 of 5.13 pM (95% CI: 4.42, 5.86) measured by a glucocorticoid receptor reporter cell line assay, while that of fresh drug was 6.89 pM (95% CI: 3.77, 12.99) (FIG. 1D).

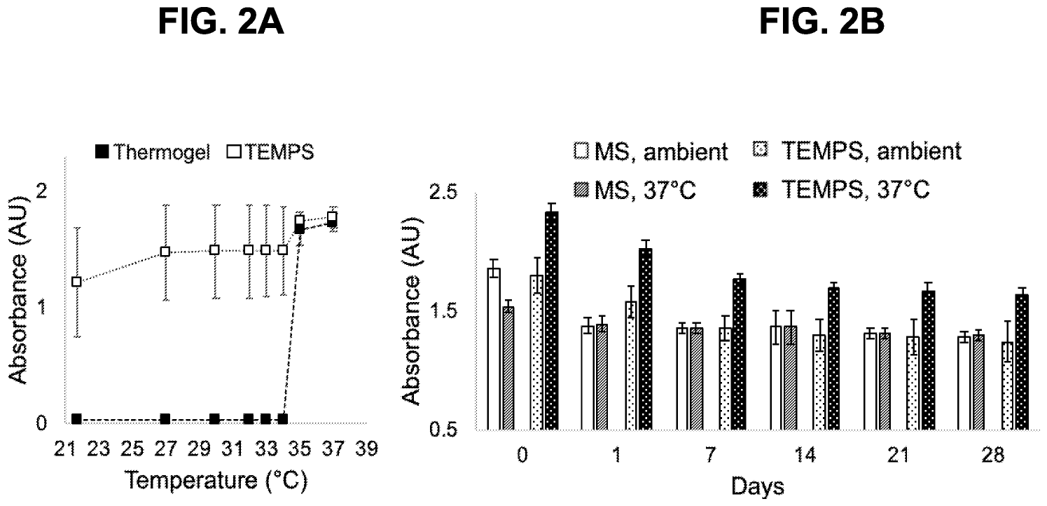

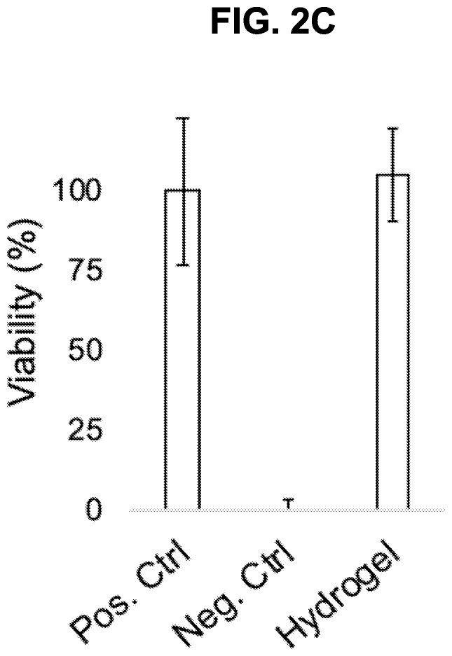

This bioactive steroid is intended for localized delivery to the paranasal sinuses via a biocompatible, reversible thermoresponsive hydrogel. Accordingly, a PEG-pNIPAAm thermogel formulation with an LCST of 35° C. was selected for the thermoresponsive matrix. The presence of drug-loaded MSs did not alter the LCST, as shown in FIG. 2A, although MSs caused a higher baseline absorbance value in the clear gel. Importantly, the reversible phase change of TEMPS, as compared to MSs alone, was demonstrated by repeat absorbance measurements while the temperature was fluctuated between ambient and body temperature over 28 days (FIG. 2B). Additionally, cytocompatibility of the PEG-pNIPAAm gel with a sinonasal epithelium cell line, RPMI 2650, was tested. After 24 hours of incubation with the gel, cellular viability was comparable to that of control cells (FIG. 2C).

Sustained, Local Steroid Release from TEMPS Reduces Sinonasal Inflammation

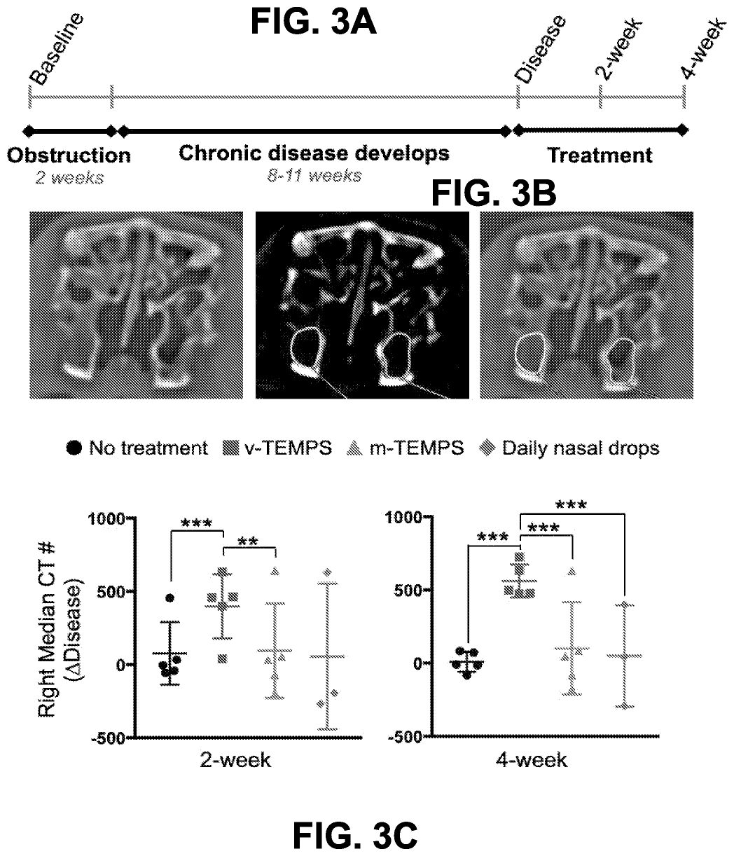

Efficacy of the extended steroid release from TEMPS was investigated in a CRS rabbit model following the timeline in FIG. 3A and using microCT measurements as depicted in FIG. 3B. Induction of disease to the left sinus cavity resulted in a significant increase in opacification between baseline and disease timepoints bilaterally. To increase disease opacification measurements, disease induction was repeated in 6 subjects using a modified timeline. Furthermore, to account for variability in disease induction results, the change in CT # from disease to the 2- or 4-weeks treatment timepoints was evaluated. On the control (right) side, change in CT # of the v-TEMPS group was significantly increased compared to no treatment (p<0.001) and m-TEMPS (p<0.020) at 2-weeks and no treatment, m-TEMPS and daily nasal drop groups (p<0.001) at 4-weeks (FIG. 3C). In contrast, the change in CT # at 2- and 4-weeks after treatment on the disease-induced (left) side showed no significant differences between groups.

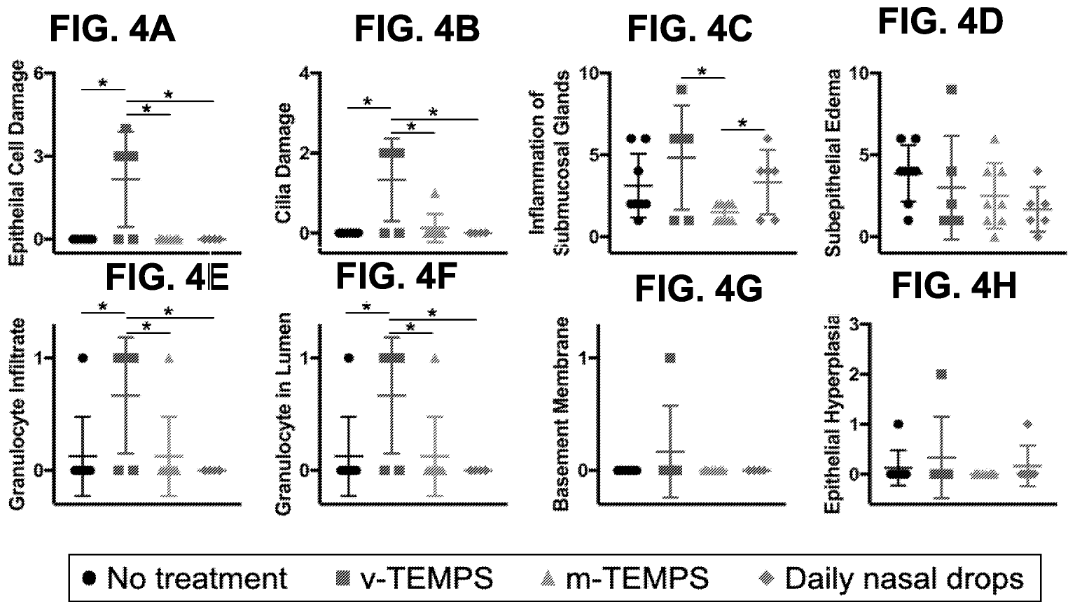

Further evaluation of the tissue response in the four treatment groups was performed using histopathology analysis and scoring of several characteristics of inflammation. On the right side, epithelial cell damage, cilia damage, granulocyte infiltrate and granulocytes in the lumen were significantly increased in the v-TEMPS group compared to the other groups (p<0.050). Additionally, inflammation of the submucosal glands was reduced in the m-TEMPS groups compared to the v-TEMPS and daily nasal drop groups (p<0.050, FIG. 4). On the left side, epithelial cell damage and cilia damage were elevated in the m-TEMPS group relative to other groups (p<0.050), but also exhibited more variability among subjects. Steroid was safely delivered from TEMPS retained in the sinus cavities for 4 weeks

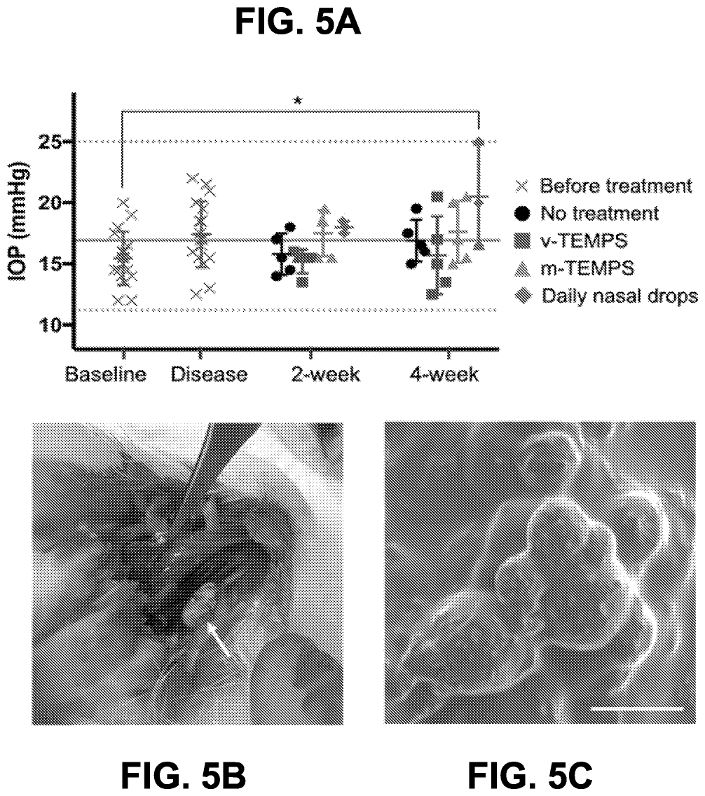

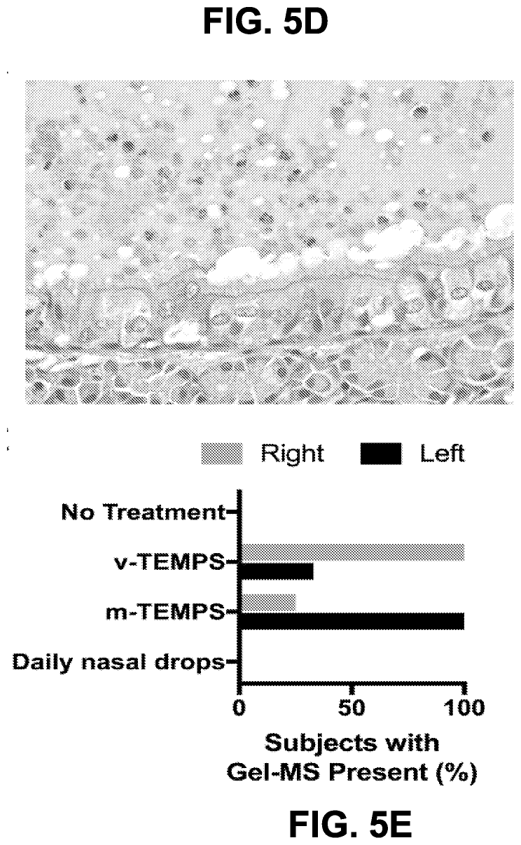

Throughout the study, bilateral IOP was measured to assess for ocular effects related to localized steroid delivery. Two weeks after treatment application, IOP was not elevated in any group. At 4 weeks, mean IOP of the daily nasal drop group was statistically elevated in comparison with baseline (p<0.050). Importantly, the m-TEMPS group did not show elevated IOP at any timepoint (FIG. 5A). Additionally, the IOP values reported here are within the range previously reported for healthy New Zealand rabbits, which over a 2 year period was 16.9 mmHg (range: 11.2-25.0, CV: 16.6%, n=125) [16]. Similarly, the overall mean IOP in this study was 16.7 mmHg (range: 12.0-25.0, CV: 15.3%, n=72).

The ability for TEMPS to be retained in the sinus cavities for 4 weeks was evaluated post-mortem. Within the sinuses of one of the subjects treated with TEMPS, a foreign material was identified (FIG. 5B). This sample was visualized using SEM and microspheres were identified (FIG. 5C). In H&E tissue sections of animals treated with TEMPS, material in the lumen was observed that displayed spherical holes that could have been the locations of polymer microspheres that dissolved during processing (FIG. 5D). The presence of this material in histological sections was noted on the left and right sides of many subjects in the subjects in both TEMPS-treated groups (FIG. 5E).

The thermoresponsive nature of the non-biodegradable hydrogel allows its application into the sinus cavities as a liquid at ambient temperature, conforming to the sinonasal epithelium as it gels. Within the nasal cavity, inspired air is quickly warmed from 25.3±2.1° C. in the nasal vestibule to 33.9±1.5° C. in the nasopharynx. Thus, for application in the sinuses, which are adjacent to the nasopharynx, TEMPS can be tuned to reversibly undergo a phase transition at a lower critical solution temperature (LCST) of 35° C. Ambient temperature saline irrigation can return the gel to a liquid state for its removal. TEMPS that was incubated at 37° C. for 4 weeks in vitro did reversibly undergo a phase change when cooled to ambient temperature at weekly intervals, as expected.

Disclosed herein is a hydrogel-based system for the paranasal sinuses that demonstrates multiple weeks of sustained, localized steroid release. Using a gel to adhere topical treatment to the sinonasal mucosa has been previously recognized (Alava et al, J. Otolaryngol.—Head Neck Surg., vol. 41, no. 3, pp. 183-188, 2012). In an uncontrolled clinical study, physicians applied a hydrophilic mometasone furoate gel to the sinuses of CRS patients up to 3 times. Drug release kinetics were not specified, and likely not sustained, which is supported by the report of modest, but temporary improvement of the diseased mucosa. Others have designed a thermoresponsive and bioadhesive gel for 4 hours of mometasone furoate release (Altuntas et al., AAPS PharmSciTech, March 2017). This gel was comprised of Pluronic® F-127 and Carbopol® 974P NF and tested for relieving allergic rhinitis in rats and promoting repair of a mucosal injury in rabbits. Another mucoadhesive in situ gel synthesized from poloxamer-407, hydroxypropyl methyl cellulose and chitosan salt demonstrated the gradual release of steroid, dexamethasone 21-phosphate disodium, for 3 days in vitro (Pandey et al, Eur. J. Pharm. Sci., vol. 96, pp. 499-507, 2017). In each of these systems, the shorter duration of drug delivery is dictated by the gel component being the rate limiting step for release. In contrast, release kinetics of TEMPS are controlled by diffusion and bulk erosion of PLGA microspheres, which can be tuned to release drugs for days, weeks, months, or even years. The pNIPAAm matrix in TEMPS (which surrounds the extended release microspheres) caused a small reduction of the initial burst release from 18.5% (MSs alone) to 11.3% (TEMPS) after 24 hours aqueous incubation, however the complete 4-week release profile was largely unchanged. It is likely that hydrophobic interactions between mometasone furoate and the pNIPAAm chains reduced the rate of initial release, but did not limit the ultimate diffusion of drug from the system. Instead, the gel component primarily serves to retain the extended release microspheres and conform to the paranasal sinus cavities.

In regard to drug loading, mometasone furoate is a lipophilic small molecule, and as such, it can be efficiently encapsulated in polymeric microparticles using a single emulsion technique. The specific PLGA MS formulation described in the Example section encapsulated 44 μg mometasone per mg MS, which was slowly released over 4 weeks. This could readily translate to a clinically relevant patient dose, in which ˜250 mg MSs would provide a sufficient quantity of drug to locally release ˜400 μg mometasone per day for 4 weeks. This dosage is equivalent to that achieved by bilateral, twice daily applications of a mometasone furoate nasal spray. Furthermore, currently available degradable sinus stents have a total loaded dose of 370 μg or 1350 μg mometasone per device and are designed for gradual release over 30 or 90 days, respectively. For patients with advanced inflammation, these stents may not provide sufficient steroid concentration for effective symptom relief. The PLGA microparticle component of TEMPS, in contrast, enables efficient surface area-to-volume loading of drug and flexible dosing by adjusting the amount of microparticles in the system.

The dosing flexibility offered by TEMPS is a product of both the amount of embedded microparticles as well as the total volume for application. The system can be applied as a thin coating along the sinus epithelium, either targeted to specific areas of inflammation or distributed throughout the sinus cavities for widespread treatment. Reported volumes of the maxillary and frontal sinuses of post-operative CRS patients and healthy individuals are in the range of 37-57 cm³. At the microparticle concentration tested in this study (50 mg/mL), a patient dose of TEMPS would equate to 5 mL, or just 9-14% of reported sinus volumes. Additionally, this dosing volume is comparable to the clinical study previously mentioned in which 2-10 mL of a hydrophilic mometasone furoate gel was applied in post-operative CRS patients and no adverse effects related to this treatment were reported.

As a preliminary measure of safety, in vitro cytocompatibility of the thermoresponsive matrix component, the PEG-pNIPAAm hydrogel, was demonstrated using a human sinonasal cell line derived from a squamous cell carcinoma of the nasal septum (RPMI 2650). While characterization of this cell line has reported several biologic differences from the primary nasal epithelium, it is the only alternative to human nasal epithelial cells (hNECs). In previous work, RPMI 2650 cells and hNECs were both used for compatibility testing of mucoadhesive and nanostructured microparticles that were developed for experimental treatment of nasal polyps. The delivery vehicle was shown to be compatible with similar trends in viability between the two cell types. Likewise, RPMI 2650 cells that were incubated with PEG-pNIPAAm hydrogel for 24 hours showed comparable viability to control cells. Furthermore, these results are consistent with reported cytocompatibility of pNIPAAm hydrogels with human conjunctival epithelial cells, as well as the numerous biomedical applications that have safely applied PEG-pNIPAAm hydrogels for in vivo testing.

Herein, the in vivo safety and retention of TEMPS was evaluated in a CRS disease model in rabbits. Rabbits are frequently used for sinusitis studies because their sinus anatomy is more similar to humans than rodents, additionally the overall size of rabbit sinuses is more conducive to studying disease or treatments, such as TEMPS. However, as is the case in humans, rabbit sinuses are not accessible through the nares without surgically removing bone. Because TEMPS is an injectable solution at room temperature and to minimize invasive procedures, vehicle-only TEMPS (v-TEMPS) and mometasone-loaded TEMPS (m-TEMPS) were injected into the left and right rabbit sinus cavities percutaneously. Recovery of the material was demonstrated in one of the subjects post-mortem, showing that the system is retained for at least 4 weeks. Notably, during those 4 weeks, intraocular pressure (IOP) was monitored in all rabbits and the subjects treated with sustained, local steroid release from m-TEMPS did not show elevated pressures. This is important given that second-generation intranasal corticosteroids, which includes mometasone furoate, have very low systemic bioavailability, however safety concerns related to high-dose and long-term steroid use, particularly for inhaled corticosteroids, still exist. As an indirect measure of systemic absorption and potential adverse effects, IOP is frequently measured in studies evaluating steroid treatments. Accordingly, IOP was monitored and, as expected, pressures across treatment groups and time remained within normal ranges previously reported for New Zealand rabbits. This is consistent with clinical studies that have reported no difference in IOP following topical delivery of mometasone furoate by nasal drops, sprays, irrigation, or stents.

These topical steroid delivery devices, along with TEMPS, are intended to reduce inflammation, which can be non-invasively monitored by computed tomography (CT) imaging. In clinical settings, CT imaging is currently recommended for the diagnosis of CRS and for pre-operative assessment of the extent and location of disease. Paranasal sinus inflammation will present on CT imaging as opacification, which is then scored using various staging systems. While scoring systems have been adapted for microCT imaging of disease in rabbit models, we reported opacification as the Hounsfield unit or CT # instead, which is a measurement of the attenuation number of the x-ray beams through the tissue of interest. The advantage of this method is that it allows for objective, blinded measurements that can be performed without prior training on assessing rabbit CT scans and could be reproducible across investigators and institutions.

In this study, the efficacy of TEMPS was measured by microCT imaging at 4 timepoints and histopathology analysis at the conclusion of the study. Comparison of the mean CT #s between the TEMPS groups revealed that treatment with m-TEMPS resulted in significantly lower sinus opacification compared to v-TEMPS at 2- and 4-weeks after application. This trend of the presence of inflammation in the v-TEMPS but not the m-TEMPS group was also observed in histopathology analyses, specifically in damage to the ciliated epithelium, submucosal gland inflammation, and presence of granulocytes. Taken together, these results suggest that the local steroid release from m-TEMPS effectively reduced inflammation. In certain embodiments, may be applied transnasally following endoscopy surgery, which we propose would be a more suitable method for application in rabbits as well. Performing this procedure in rabbits will require precise bone removal to surgically open the sinus ostia via the nares in order to access the sinus cavities while inducing as little added trauma to the surrounding anatomy.

Along with the goal of minimizing tissue trauma with TEMPS application, a minimally invasive disease model was selected as well; however, the overall disease induction was highly variable. In fact, obstructing the left ostiomeatal complex to induced disease was repeated in 6 subjects to increase the measured opacification on microCT images. While this non-infectious CRS model has shown success previously, due to the inconsistent change between baseline CT # to disease CT # and the small sample size, the effect of m-TEMPS to reduce opacification due to sinus ostium obstruction could not be evaluated. In future studies, other disease models or more complete ostium obstruction will be explored, although currently, there is no ideal preclinical model for CRS and this remains an area of continued development.

In summary, data suggest that TEMPS may provide sustained, bioactive drug delivery for reduction of sinonasal inflammation. TEMPS is cyto-compatible in vitro and is well-tolerated in rabbits in vivo. Due to its temperature-sensitive nature, TEMPS can provide apposition to unique tissue architecture, like the sinus cavities. Moreover, while the hydrogel matrix is non-biodegradable, the system is temporary and can be readily removed and reapplied as needed.

### Retinoic Acid Release

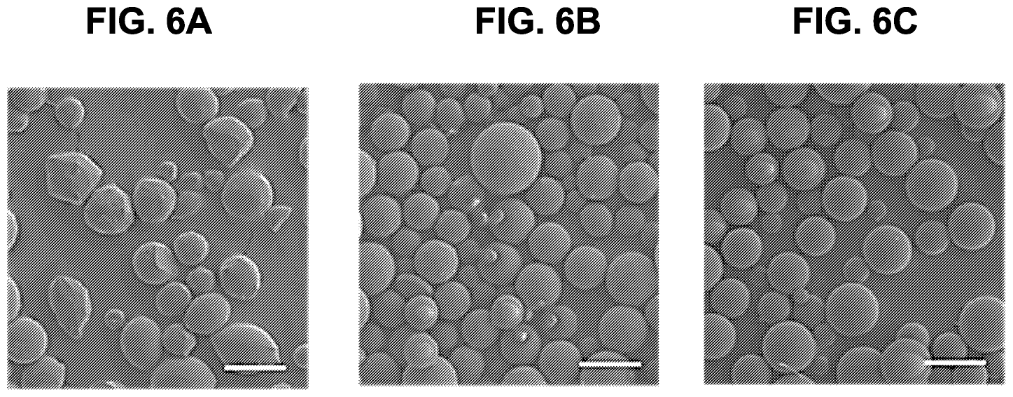

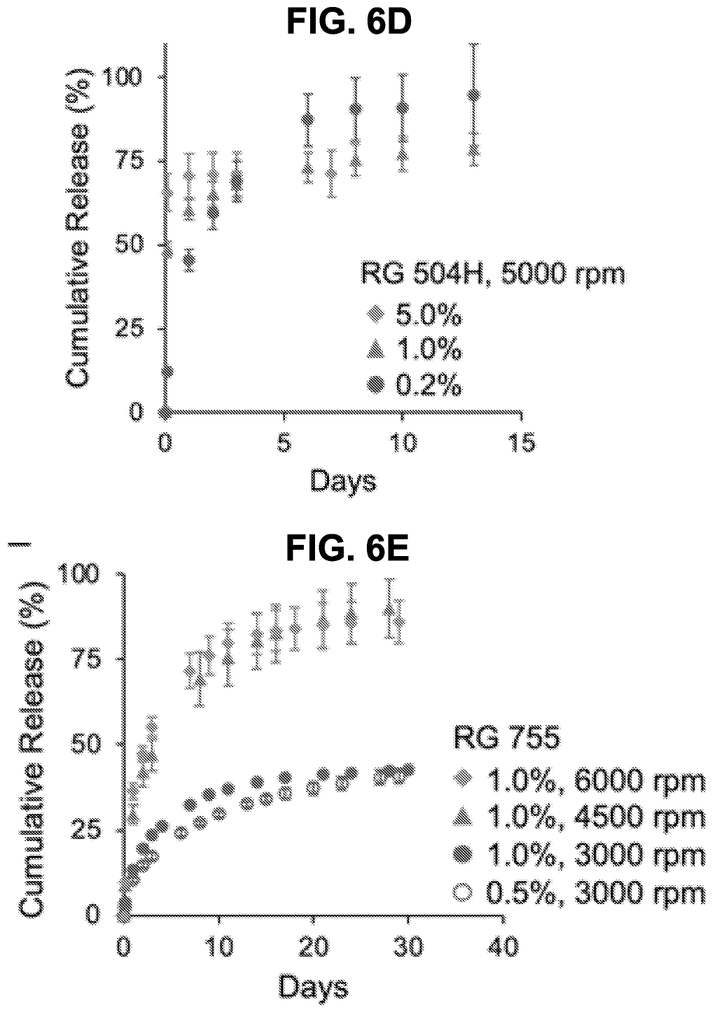

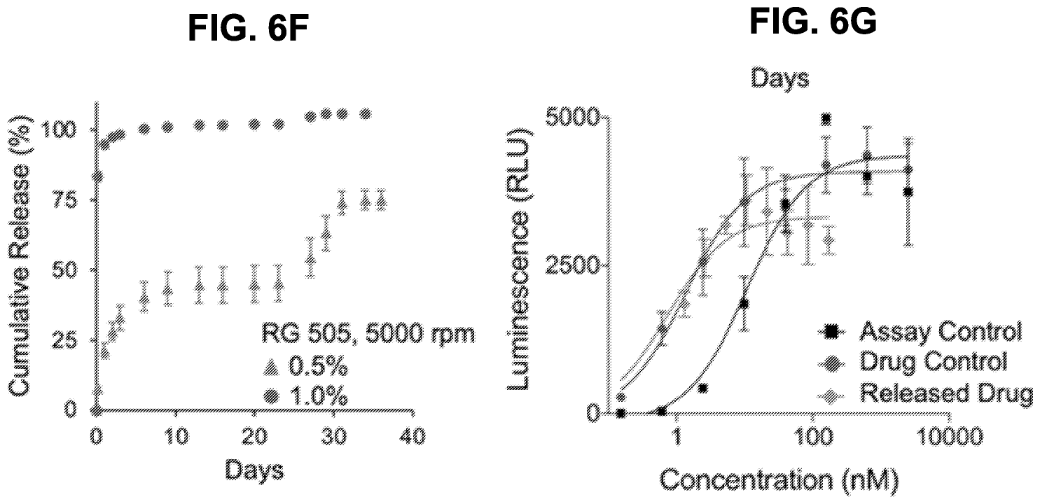

TEMPS (Thermogel, Extended-release, Microsphere-based delivery to the Paranasal Sinuses) were prepared for the sustained release of retinoic acid, a small molecule that has been shown to promote regeneration of ciliated epithelium. Microsphere fabrication parameters, including the weight of RA, homogenization speed, and properties of PLGA, were evaluated for their effect on MS size and morphology, drug loading, and release kinetics (FIGS. 6A-6G and Table 1). An interacting effect between the weight of RA and properties of PLGA was observed, where higher drug amounts resulted in irregularly shaped MSs with a burst release. By decreasing the amount of drug and/or using PLGA was higher lactic acid content, the burst release was decreased. PLGA MS formulations were developed that achieved sustained release of RA for at least 30 days in vitro (FIGS. 6E and 6F). Moreover, the released RA was shown to maintain its bioactivity using a receptor-binding reporter cell assay (FIG. 6G).

TABLE 1
Microsphere fabrication parameters and characterization

| | | Homogen- | | | Encapsu- |
|---|---|---|---|---|---|
| | RA | ization | Average | | lation |
| | weight | Speed | Diameter | Loading | Efficiency |
| PLGA | (w/w)* | (rpm) | (μm) | (μg/mg) | (%) |
| RG 504H | 5.0% | 5000 | 11.2 ± 5.1 | 53.7 | 107 |
| | 1.0% | 5000 | 7.3 ± 2.3 | 9.4 | 94 |
| | 0.2% | 5000 | 6.6 ± 2.1 | 1.5 | 76 |
| RG 755 | 0.5% | 3000 | 24.3 ± 7.0 | 4.0 | 79 |
| | 1.0% | 3000 | 18.5 ± 5.4 | 7.4 | 74 |
| | 1.0% | 4500 | 9.5 ± 3.1 | 8.8 | 78 |
| | 1.0% | 6000 | 9.1 ± 5.0 | 5.9 | 59 |
| RG 505 | 0.5% | 3000 | 20.0 ± 6.2 | 3.4 | 68 |
| | 1.0% | 3000 | 20.4 ± 7.7 | 8.3 | 83 |

*weight of RA (mg) to weight of PLGA (mg)

TABLE 2
Properties of PLGA used to fabricated RA MSs

| | Molecular | Viscosity | | |
|---|---|---|---|---|
| PLGA | weight (Da) | (dL g⁻¹) | LA:GA | End-group |
| RG 504H | 38,000-54,000 | 0.45-0.60 | 50:50 | acid |
| RG 505 | 54,000-69,000 | 0.61-0.74 | 50:50 | ester |
| RG 755 | 50,000-75,000 | 0.50-0.70 | 75:25 | ester |

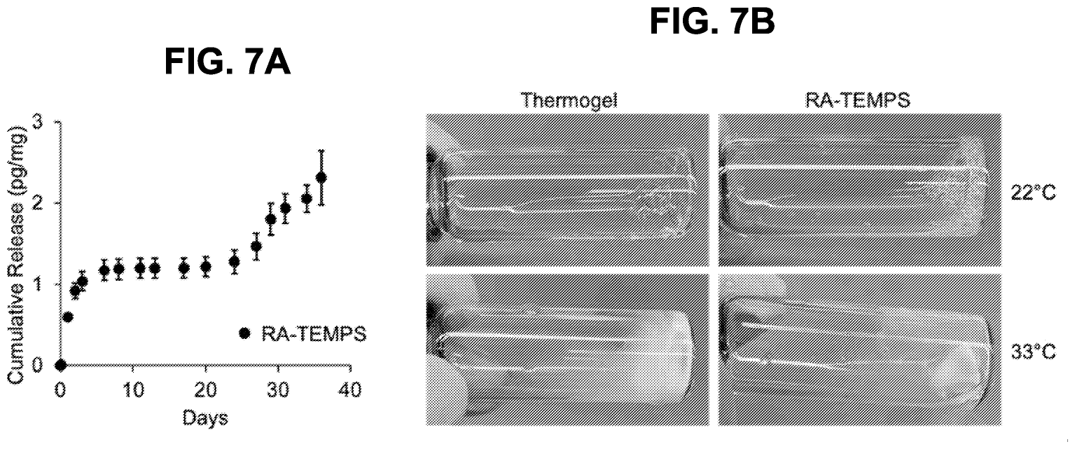

When PLGA MSs loaded with RA were mixed in the thermogel, forming RA-TEMPS, the release profile of RA was similar to that of MSs alone (FIG. 7A and FIG. 6F “0.5%”). The gelation temperature of RA-TEMPS was also consistent with thermogel alone at 33° C. (FIG. 7B).

### CCL22 Release

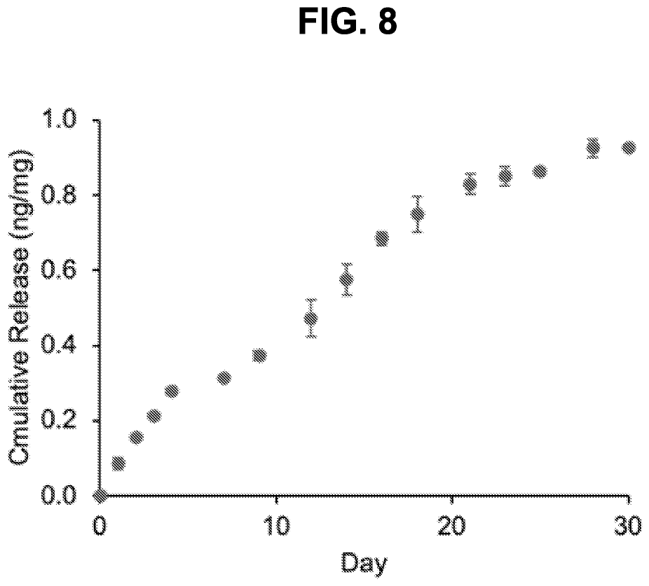

TEMPS has also been developed for the sustained release of a protein, C-C motif chemokine ligand 22 (CCL22). CCL22-loaded microspheres were embedded in the thermogel, and the resulting in vitro drug release was sustained over 30 days (FIG. 8).

Cilia Compatibility with Thermogel

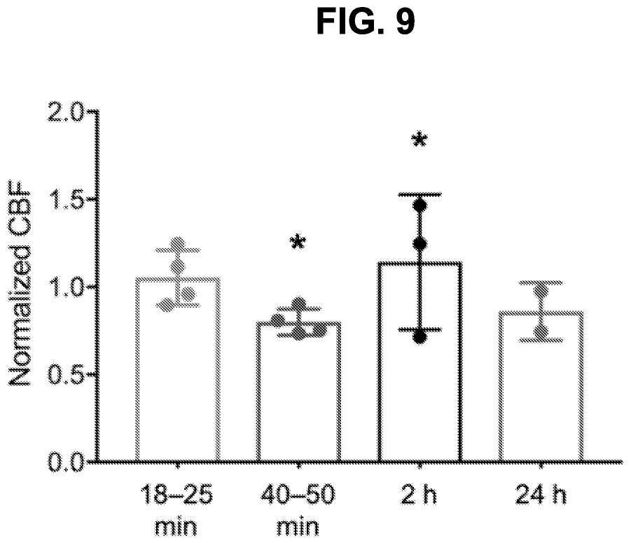

Since in certain embodiments TEMPS may be in constant contact with the sinonasal epithelium, compatibility of the thermogel with ciliated nasal epithelium collected from healthy volunteers was assessed by measuring changes in cilia beat frequency (CBF) following ex vivo incubation. A short duration of incubation (18-25 min) did not significantly affect CBF while incubation for 40-50 min or 2 h did demonstrate a significant change in CBF from control samples (p<0.05), although the differences were nominally small (FIG. 9). Moreover, 2 re-ciliated samples were evaluated for 24 h incubation with thermogel and no differences in CBF from the control were observed. Overall, the ex vivo cilia samples remained motile after incubation with the thermogel.

### Freeze-Dried TEMPS for Clinical Translation

A freeze-dried TEMPS formulation (“FD-TEMPS”) was developed to enable shelf stability of the combined MSs and thermogel. Additionally, FD-TEMPS could be applied to the sinonasal mucosa in its dried format where contact with the mucus layer that consists of ˜95% water (w/w) could rehydrate the material immediately prior to gelation. The thermoresponsive behavior and drug release of FD-TEMPS were developed for consistency with its original formulation and proof-of-concept experiments were performed to demonstrate in situ rehydration, gelation, and mucoadhesion.

FD-TEMPS was prepared as follows. The pNIPAAm thermogel was prepared by aqueous free radical polymerization of N-isopropylacrylamide with the addition of polyethylene glycol (PEG) using similar methods as previously reported. For freeze-drying studies, the thermogel was prepared with 0-5% (w/w) PEG of 2000-8000 molecular weight (MW) or 5.6% PEG of 200 MW. Microspheres were mixed with the thermogel immediately prior to freezing in glass vials or Eppendorf tubes at −80° C. for 24 h. The vial caps were replaced with permeable kimwipe cover and the frozen vials were transferred to the freeze-drier, operating at <100 mTorr for 3 days with the condenser held at −50° C. The freeze-dried gels were stored in a desiccator at ambient temperature until characterization.

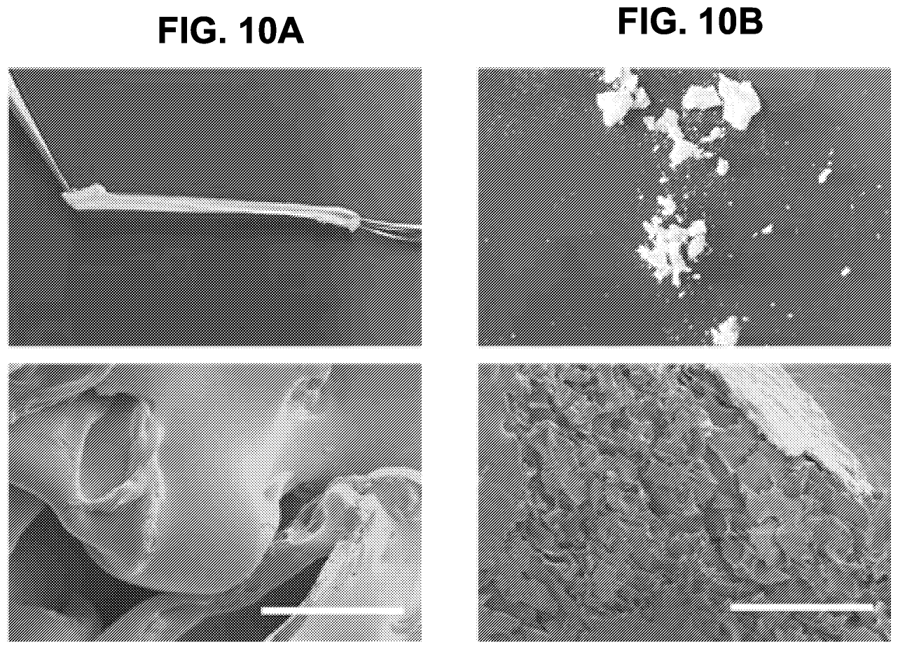

In order to obtain a suitable dry, powder-like product, the thermogel formulation had to be modified. By replacing the low molecular weight polyethylene glycol (PEG) (FIG. 10A and Table 3 “Control”) with a higher molecular weight PEG (FIG. 10A and Table 3 “5% PEG2000”), the freeze-dried gel exhibited a brittle texture and was able to be crushed (FIG. 10B). Additional, scanning electron microscopy (SEM) revealed the formation of pores that indicate the removal of water during the freeze-drying process. The 5% PEG2000 formulation also exhibited a glass transition temperature of the freeze-concentrated solution (T_{g}′), a critical temperature below which the product must be maintained during freeze drying (Table 3).

TABLE 3
Thermogel transition temperatures

| | | Sol-gel | | | |
|---|---|---|---|---|---|
| Thermogel | T_{g}′ | endotherm | LCST₀ | LCST_{FD-H2O} | LCST_{FD-SNF} |
| Formulation | (° C.) | (° C.) | (° C.) | (° C.) | (° C.) |
| pNIPAAm only | −9.9 | 32.5 | 33.0 | 33.0 | 30.4 |
| Control | N/A | 32.0 | 32.7 | 33.0 | 30.3 |
| 5.0% PEG2000 | −9.7 | 32.6 | 32.7 | 33.0 | 30.4 |

T_{g}′ = glass transition temperature of freeze concentrate.
N/A = T_{g}′ was not detected.
T_{g}′ and sol-gel endotherm were measured by DSC.
LCST₀, LCST_{FD-H2O}, and LCST_{FD-SNF} = lower critical solution temperature of original, freeze-dried and rehydrated in water, or SNF, respectively.

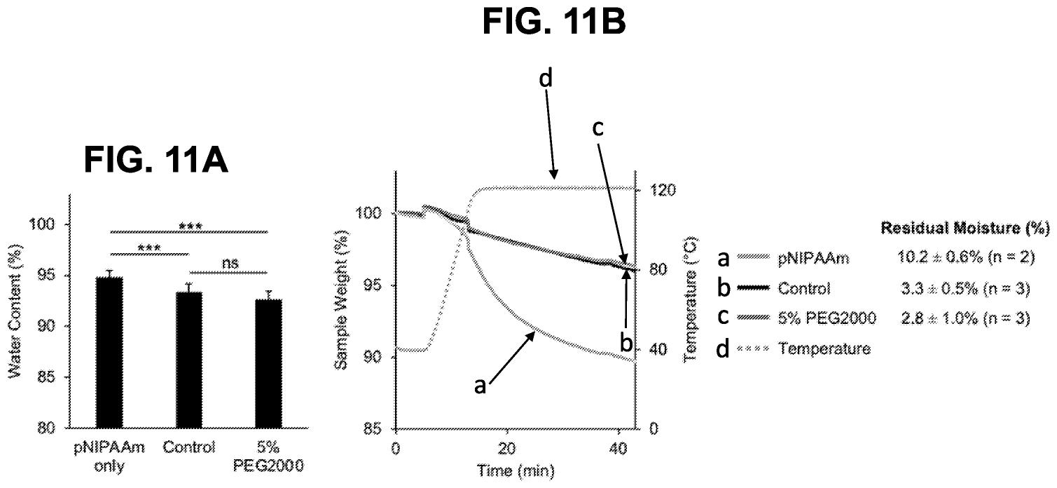

The freeze-dried thermogel was also evaluated for the residual moisture content, which must be minimized to enhance shelf stability. Formulations containing PEG were found to have <4% residual moisture (FIG. 11B), which was determined by thermal gravimetric analysis.

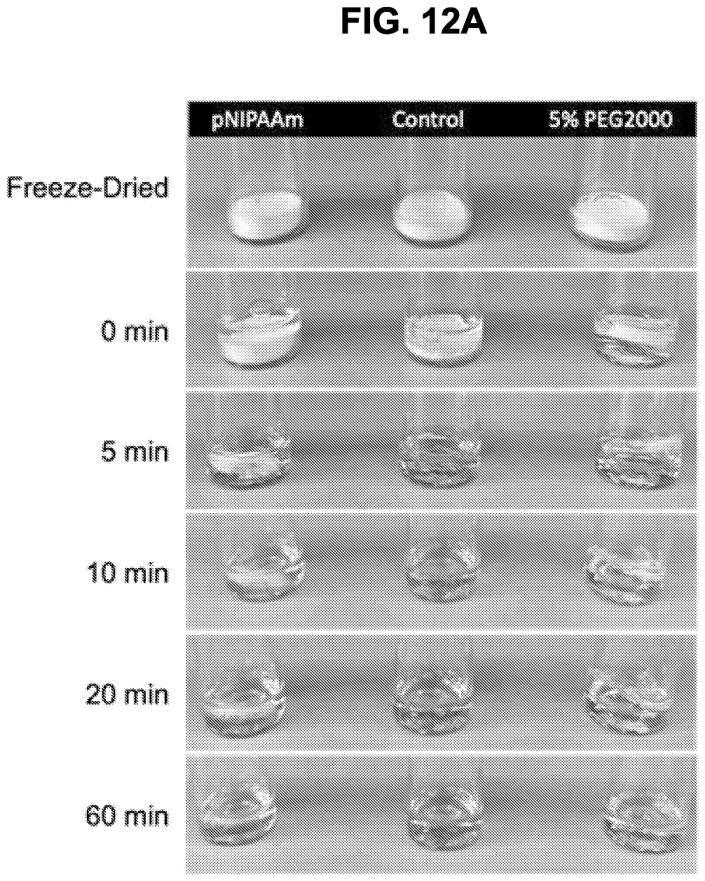

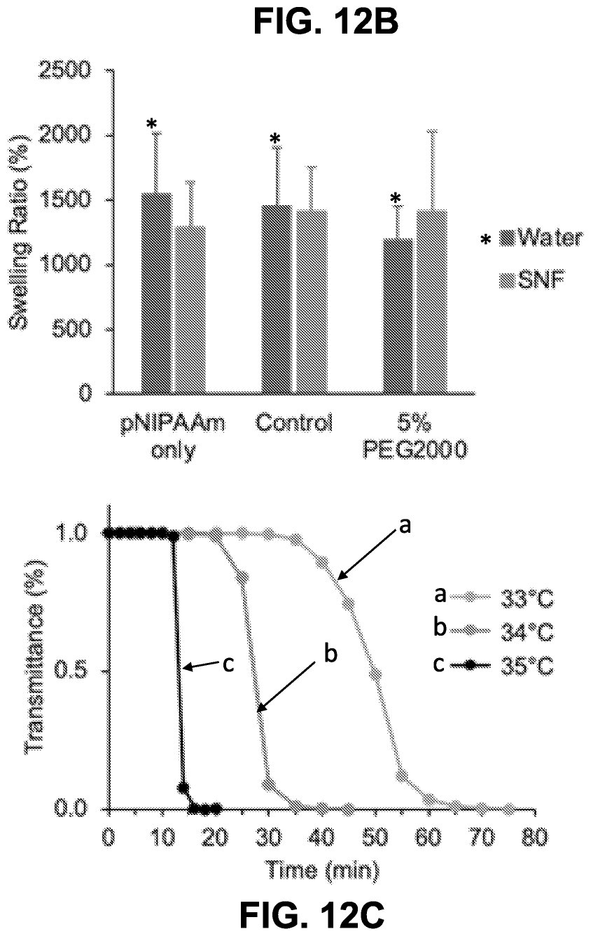

Freeze-dried 5% PEG2000 gel rehydrated in ˜60 minutes (FIG. 12A) and was shown to swell by 1000% when submerged in water or a simulated nasal fluid (SNF) (FIG. 12B). Gelation of the rehydrated 5% PEG2000 gel was demonstrated at physiological temperatures of the sinuses (FIG. 12C).

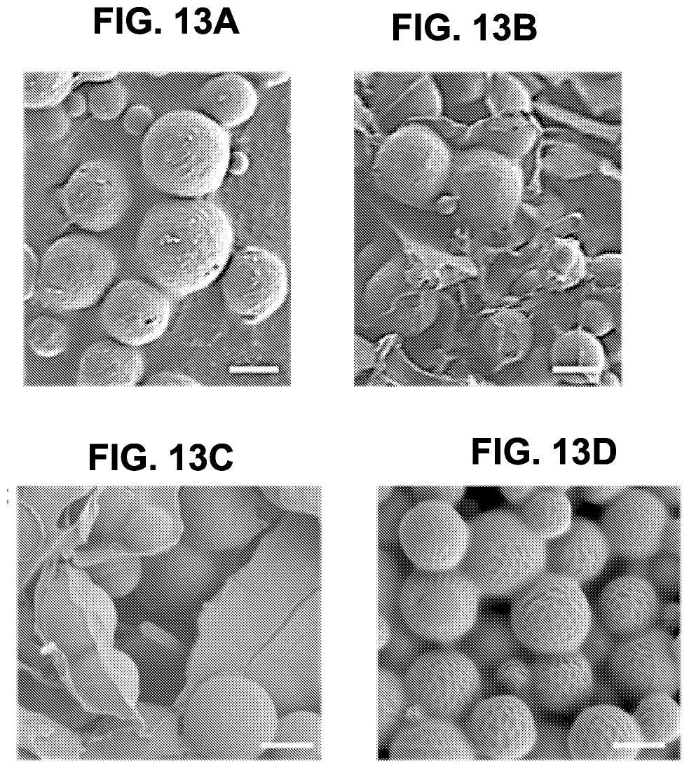

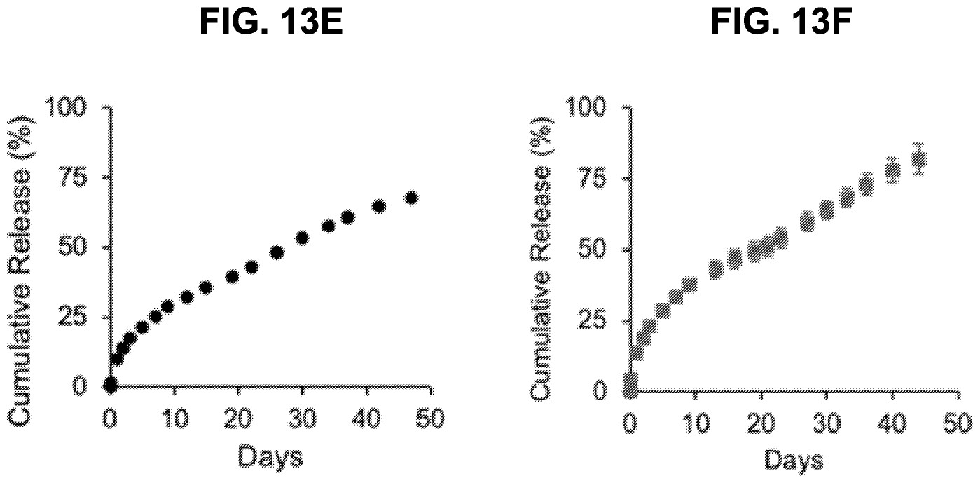

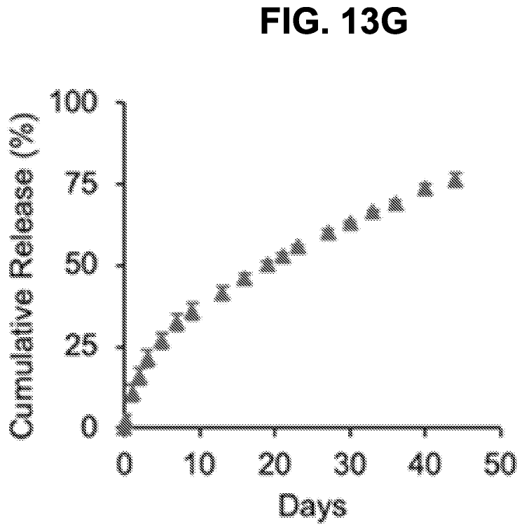

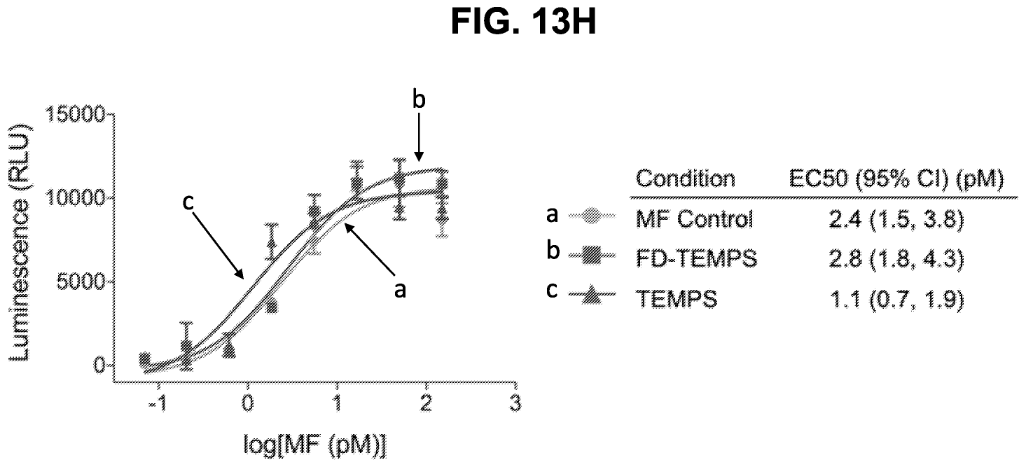

Shelf-stability of FD-TEMPS was evaluated by measuring drug release kinetics following storage at ambient conditions. Release of the corticosteroid mometasone furoate was evaluated as it is the standard of care for CRS. The storage state (freeze-dried or hydrated) had an effect on the surface morphology of the microspheres but, unexpectedly, did not affect the release kinetics of bioactive drug. As shown in FIG. 13A, mometasone-loaded microspheres had a smooth, spherical morphology that was maintained when combined with the thermogel and freeze-dried (FIG. 13B). The surface morphology remained unchanged when the freeze-dried formulation was stored at room temperature for 3 weeks (FIG. 13C). However, when stored in its hydrated state (i.e. the microspheres were combined with the thermogel and were not freeze-dried), erosion of the microsphere surface was apparent (FIG. 13D). Notably, the change in surface morphology was not associated with different release kinetics of mometasone. The release profiles for fresh FD-TEMPS, 3-weeks stored FD-TEMPS, and 3-weeks stored TEMPS were very similar (FIGS. 13E-13G). Additionally, the bioactivity of mometasone released from the two stored formulations was similar to that of control mometasone that was reconstituted the same day as the assay (FIG. 13H).

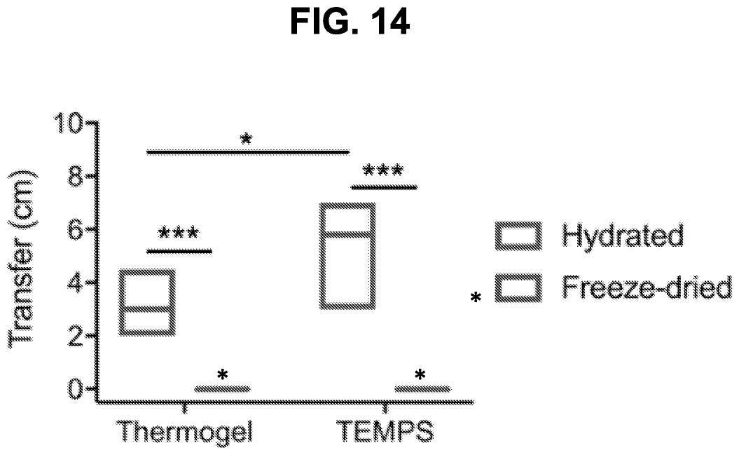

The ability of FD-TEMPS to remain in place after application to a simulated mucosal surface was evaluated by measuring the transfer distance on an agar and mucin plate. In its hydrated state, both the thermogel and TEMPS were displaced several centimeters from their application site. The presence of MSs in TEMPS also increased the transfer distance. In contrast, the freeze-dried thermogel and FD-TEMPS remained in place after application (FIG. 14). Importantly, the material remained in place 6 hours later.

In view of the many possible embodiments to which the principles of the disclosed invention may be applied, it should be recognized that the illustrated embodiments are only preferred examples of the invention and should not be taken as limiting the scope of the invention.

1. A paranasal and/or nasal delivery system comprising:
a thermoresponsive gel; and
a plurality of microparticles comprising a therapeutic amount of at least one paranasal and/or nasal condition-treating therapeutic agent,
wherein the microparticles are included in the thermoresponsive gel.

2. The paranasal and/or nasal delivery system of claim 1, wherein the therapeutic agent is an intranasal corticosteroid, an antibiotic, a biologic, an immunosuppressant, or a retinoid.

3. The paranasal and/or nasal delivery system of claim 1, wherein the therapeutic agent is mometasone furoate, fluticasone propionate, ciclesonide, fluticasone furoate, dexamethasone, flunisolide, triamcinolone acetonide, beclomethasone dipropionate, or budesonide.

4. The paranasal and/or nasal delivery system of claim 1, wherein the therapeutic agent is mometasone furoate.

5. The paranasal and/or nasal delivery system of claim 1, wherein the therapeutic agent is retinoic acid.

6. The paranasal and/or nasal delivery system of claim 1, wherein the therapeutic agent is CCL22.

7. The paranasal and/or nasal delivery system of claim 1, wherein the thermoresponsive gel comprises poly(N-isopropylacrylamide).

8. The paranasal and/or nasal delivery system of claim 1, wherein the thermoresponsive gel comprises a mixture of N-isopropylacrylamide and a hydrophilic polymer.

9. The paranasal and/or nasal delivery system of claim 8, wherein the hydrophilic polymer is polyethylene glycol.

10. The paranasal and/or nasal delivery system of claim 1, wherein the microparticles further comprise a biodegradable polymer.

11. The paranasal and/or nasal delivery system of claim 1, wherein microparticles further comprise a biodegradable polymer selected from polyglycolide (PGA), poly(lactic acid) (PLA), poly(lactic-co-glycolic acid) (PLGA), or any combination thereof.

12. The paranasal and/or nasal delivery system of claim 11, wherein the biodegradable polymer comprises PLGA.

13. The paranasal and/or nasal delivery system of claim 1, wherein the therapeutic agent is homogeneously dispersed within the microparticles.

14. The paranasal and/or nasal delivery system of claim 1, wherein the microparticles are homogeneously dispersed within the thermoresponsive gel.

15. The paranasal and/or nasal delivery system of claim 1, wherein the thermoresponsive gel is non-biodegradable.

16. The paranasal and/or nasal delivery system of claim 1, wherein the system is in a freeze-dried form.

17. The paranasal and/or nasal delivery system of claim 1, wherein the system is in a lyophilized powder form.

18. A method for treating a paranasal and/or nasal condition in a subject comprising administering to the subject in need thereof the paranasal and/or nasal delivery system of claim 1, wherein the paranasal and/or nasal delivery system gels following administration.

19. The method of claim 18, wherein the paranasal and/or nasal delivery system is administered topically to at least one paranasal and/or nasal cavity.

20. The method of claim 19, wherein the paranasal and/or nasal delivery system is administered transnasally via a catheter.

21. The method of claim 18, wherein the paranasal and/or nasal delivery system gels to form a coating along the sinonasal epithelium.

22. The method of claim 18, wherein the paranasal and/or nasal delivery system is targeted to a specific area of inflammation.

23. The method of claim 18, wherein the paranasal and/or nasal delivery system is distributed throughout the sinus cavities for widespread treatment.

24. The method of claim 18, wherein the paranasal and/or nasal delivery system is administered following transnasal endoscopic surgery.

25. The method of claim 18, wherein the method provides sustained release of the therapeutic agent for at least 30 days.

26. The method of claim 18, wherein the paranasal and/or nasal condition is chronic rhinosinusitis, acute rhinosinusitis, recurrent acute rhinosinusitis, allergic rhinitis or an olfactory disorder.

27. The method of claim 18, wherein the paranasal and/or nasal condition is chronic rhinosinusitis.

28. The method of claim 18, further comprising removing the gelled paranasal and/or nasal delivery system via room temperature saline irrigation.

29. The method of claim 18, wherein the microparticles are mixed with the thermoresponsive gel immediately prior to administering to the subject.

30. A method comprising administering to a subject the paranasal and/or nasal delivery system of claim 1, wherein the paranasal and/or nasal delivery system gels following administration and promotes regeneration of ciliated nasal epithelium.

31. The method of claim 30, wherein the paranasal and/or nasal delivery system is administered topically to at least one paranasal and/or nasal cavity.

32. The method of claim 30, wherein the paranasal and/or nasal delivery system is administered transnasally via a catheter.

33. The method of claim 30, wherein the paranasal and/or nasal delivery system gels to form a coating along the sinonasal epithelium.
